(19) United States
(12) Patent Application Publication
(10) Pub. No.: US 2026/0022342 A1
(43) Pub. Date: Jan. 22, 2026

(54) METHODS AND SYSTEMS FOR DEVELOPING MEDIA FOR EXTRACELLULAR VESICLE PRODUCTION

(71) Applicants: FUJIFILM Holdings America Corporation, Valhalla, NY (US); FUJIFILM Irvine Scientific, Inc., Santa Ana, CA (US)

(72) Inventors: Ken NARUSE, Santa Ana, CA (US); Cassandra WRIGHT BELLVER, Santa Ana, CA (US); Alison FUJII, Santa Ana, CA (US); Aaron FULTON, Santa Ana, CA (US); Richard A. FESTA, Santa Ana, CA (US); Matthew TENORIO, Santa Ana, CA (US); David T. HO, Santa Ana, CA (US); Nisa RENAULT, Madison, WI (US); Michele HAMRICK, Madison, WI (US); Jacquelyn WONG, Madison, WI (US); Robert E. NEWMAN, Santa Ana, CA (US); Maria Katerina R. VILLAFUERTE, Santa Ana, CA (US)

(73) Assignees: FUJIFILM Holdings America Corporation, Valhalla, NY (US); FUJIFILM Irvine Scientific, Inc., Santa Ana, CA (US)

(21) Appl. No.: 18/868,535

(22) PCT Filed: May 24, 2023
(86) PCT No.: PCT/US2023/023378

§ 371 Date: Nov. 22, 2024

Related U.S. Application Data
(60) Provisional application No. 63/345,223, filed on May 24, 2022.
(60) Provisional application No. 63/467,919, filed on May 19, 2023.

(51) Int. Cl. C12N 5/0775; C12M 1/34; C12M 1/36; C12N 5/074; G01N 33/569

(52) U.S. Cl. CPC C12N 5/0662; C12M 41/36; C12M 41/48; C12N 5/0696; G01N 33/56966; G01N 2333/70596

(57) ABSTRACT

The present disclosure relates generally to methods and systems for the analysis, development, testing, and/or optimization, of culture media and/or culture conditions for extracellular vesicle production; culture media analyzed, developed, tested, and/or optimized via such assays; and to extracellular vesicles and compositions comprising extracellular vesicles generated using such culture media.

### FIELD OF THE INVENTION

The present disclosure relates, generally, to methods and systems for the analysis, development, testing, and/or optimization, of culture media (and/or culture conditions) for extracellular vesicle production. The present disclosure further relates to culture media analyzed, developed, tested, and/or optimized, via such extracellular vesicle characterization assays; and to extracellular vesicles and extracellular vesicle-containing compositions generated using such culture media.

### BACKGROUND INFORMATION

Cells, including those in in vitro or ex vivo culture, secrete a large variety of molecules and biological factors (collectively known as a secretome) into the extracellular space. See Vlassov et al. (Biochim Biophys Acta, 2012; 940-948). As part of the secretome, various bioactive molecules are secreted from cells within membrane-bound extracellular vesicles, such as exosomes. Extracellular vesicles are capable of altering the biology of other cells through signaling, or by the delivery of their cargo (including, for example, proteins, lipids, and nucleic acids). The cargo of extracellular vesicles is encased in a membrane which, amongst others, allows for specific targeting (e.g., to target cells) via specific markers on the membrane; and increased stability during transport in biological fluids, such as through the bloodstream or across the blood-brain-barrier (BBB).

Exosomes exert a broad array of important physiological functions, e.g., by acting as molecular messengers that traffic information between different cell types. For example, exosomes deliver proteins, lipids and soluble factors including RNA and microRNAs which, depending on their source, participate in signaling pathways that can influence apoptosis, metastasis, angiogenesis, tumor progression, thrombosis, immunity by directing T cells towards immune activation, immune suppression, growth, division, survival, differentiation, stress responses, apoptosis, and the like. See Vlassov et al. (Biochim Biophys Acta, 2012; 940-948). Extracellular vesicles may contain a combination of molecules that may act in concert to exert particular biological effects. Exosomes incorporate a wide range of cytosolic and membrane components that reflect the properties of the parent cell. Therefore, the terminology applied to the originating cell can in some instances be used as a simple reference for the secreted exosomes.

Extracellular vesicles, such as exosomes, have considerable potential for use as an effective cell-free therapy (with attendant benefits such as improved convenience, stability, and operator handling), for the treatment of a variety of diseases, including, for example, cancer, heart disease, and inflammation. However, there currently is a need for methods that improve the yield and/or quality of extracellular vesicles from extracellular vesicle-producing cells; and for methods to establish and optimize production and purification processes for therapeutic extracellular vesicles.

### SUMMARY OF THE INVENTION

The present disclosure addresses the above-described limitations in the art, by providing methods and systems for the analysis, development, testing, and/or optimization, of culture media (and/or culture conditions) for extracellular vesicle production; culture media analyzed, developed, tested, and/or optimized, via such assays; and to extracellular vesicles and extracellular vesicle-containing compositions comprising extracellular vesicles produced using such culture media.

For example, in some embodiments, the present disclosure provides high-throughput methods for one or more of cell culture; media exchange; vesiculation; analysis of cell growth and/or viability; analysis of extracellular vesicle production and secretion; and characterization of extracellular vesicles, thereby providing improved methods and systems for the analysis, development, testing, and/or optimization, of culture media (and/or culture conditions) for extracellular vesicle production (such as by allowing an increase in the number of test samples, and/or a reduction in the length of time).

Non-limiting embodiments of the disclosure include as follows:

[1]A high-throughput method for analyzing, developing, and/or optimizing, a culture medium for extracellular vesicle production, said method comprising: (a) culturing cells in a first culture medium, wherein cell division occurs during the culturing, and wherein said culturing is performed in multiplicate; (b) after step (a), removing said first culture medium from the multiplicate cell cultures, adding different candidate vesiculation culture media to different cell cultures amongst said multiplicate cell cultures, and further culturing the multiplicate cell cultures to produce conditioned media containing extracellular vesicles; (c) recovering, from the multiplicate cell cultures, either the conditioned media, the cells after the culturing of step (b), or both; and (d) analyzing at least one property of either the extracellular vesicles in the recovered conditioned media, the recovered cells, or both, wherein at least one of steps (a)-(d) is at least partially automated.

[2] The method of [1], wherein at least one of steps (a)-(c) is semi-automated.

[3] The method of [1], wherein at least one of steps (a)-(c) is fully-automated.

[4] The method of [1], wherein each of steps (a)-(c) are semi-automated or fully-automated.

[5] The method of any one of [1]-[4], wherein at least one of steps (a)-(c) is performed using an automated liquid handler.

[6] The method of [5], wherein each of steps (a)-(c) are performed using an automated liquid handler.

[7] The method of any one of [1]-[6], wherein said cells are iPSC-derived cells.

[8] The method of [7], wherein the culturing is two-dimensional cell culture.

[9] The method of [8], wherein said two-dimensional cell culture comprises culturing said cells on a surface of a culture vessel.

[10] The method of [9], wherein said culture vessel surface is coated with a substance to promote cell adhesion.

[11] The method of [10], wherein said substance to promote cell adhesion is vitronectin or fibronectin.

[12] The method of any one of [1]-[11], wherein the multiplicate cell cultures are cultured within one or more multi-well plates or micro-well plates.

[13] The method of any one of [1]-[12], wherein said method further comprises blending or combining two or more culture media, and/or adding one or more additives or supplements to one or more culture media, to produce a panel of different candidate vesiculation culture media.

[14] The method of [13], wherein the panel of different candidate vesiculation culture media is produced using an automated liquid handler.

[15] The method of [14], wherein the panel of candidate vesiculation culture media is produced by blending or combining two or more culture media from an initial selection of at least five different culture media.

[16] The method of [15], wherein the panel of candidate vesiculation culture media is produced by blending or combining two or more culture media from an initial selection of at least ten different culture media.

[17] The method of [16], wherein the panel of candidate vesiculation culture media is produced by blending or combining two or more culture media from an initial selection of at least twenty different culture media.

[18] The method of [17], wherein the panel of candidate vesiculation culture media is produced by blending or combining two or more culture media from an initial selection of at least fifty different culture media.

[19] The method of any one of [14]-[18], wherein the panel of candidate vesiculation culture media comprises at least ten candidate vesiculation culture media.

[20] The method of [19], wherein the panel of candidate vesiculation culture media comprises at least twenty candidate vesiculation culture media.

[21] The method of [20], wherein the panel of candidate vesiculation culture media comprises at least thirty candidate vesiculation culture media.

[22] The method of [21], wherein the panel of candidate vesiculation culture media comprises at least fifty candidate vesiculation culture media.

[23] The method of [22], wherein the panel of candidate vesiculation culture media comprises at least a hundred candidate vesiculation culture media.

[24] The method of any one of [1]-[23], wherein said cells comprise progenitor cells.

[25] The method of any one of [1]-[24], wherein said cells have previously been frozen.

[26] The method of any one of [1]-[25], wherein the at least one property of the recovered cells from step (c) analyzed is selected from the group consisting of the cell number, cell viability, cell density, morphologies of the cells, identity of the cells, karyotype of the cells, transcriptome of the cells, hypertrophy, cell health, cell adhesion, cell physiology, and/or ATP content.

[27] The method of [26], wherein the at least one property is selected from the group consisting of cell number and cell viability.

[28] The method of [27], wherein cell number and/or cell viability is measured using an automated cell counter.

[29] The method of [27] or [28], wherein cell number and/or cell viability is determined by staining cells with at least one dye.

[30] The method of [29], wherein the cells are stained with acridine orange and/or propidium iodide.

[31] The method of any one of [1]-[25], wherein the at least one property of the extracellular vesicles analyzed is total extracellular vesicle number, extracellular vesicle number per cell, extracellular vesicle concentration, extracellular vesicle size, extracellular vesicle size distribution, protein concentration, protein profile concentration, RNA profile, potency, or marker expression.

[32] The method of [31], wherein the at least one property of the extracellular vesicles analyzed is selected from total extracellular vesicle number, extracellular vesicle number per cell, extracellular vesicle size, and marker expression.

[33] The method of [32], wherein the total extracellular vesicle number and/or extracellular vesicle number per cell is determined by measuring the expression of at least one marker present on extracellular vesicles.

[34] The method of [33], wherein the marker is a tetraspanin.

[35] The method of [34], wherein the tetraspanin is selected from the group consisting of CD9, CD63 and CD81.

[36] The method of [35], wherein the tetraspanin is CD63.

[37] The method of any one of [32]-[36], wherein the marker is detected using an immunoassay.

[38] The method of [37], wherein the immunoassay is ELISA.

[39] The method of [38], wherein the ELISA is a Tim4-capture ELISA.

[40] The method of [39], wherein the ELISA is a Tim4-capture ELISA that detects CD63 expression.

[41] The method of any one of [26]-[40], wherein the measurement of the at least one property is at least partially automated.

[42] The method of [31], wherein the at least one property of the extracellular vesicles analyzed is analyzed at the single extracellular vesicle level.

[43] The method of [42], wherein the at least one property analyzed at the single extracellular vesicle level is marker expression.

[44] The method of [42] or [43], wherein the analysis is conducted using super resolution microscopy.

[45] The method of [44], wherein the super resolution microscopy is Direct Stochastic Optical Reconstruction Microscopy (dSTORM).

[46] The method of any of [43]-[45], wherein the marker expression analyzed comprises analysis of the expression of at least one tetraspanin.

[47] The method of [46], wherein the tetraspanin is selected from the group consisting of CD9, CD63 and CD81.

[48] The method of any one of [42], [44] and [45], wherein the size of individual extracellular vesicles is analyzed.

[49] The method of any one of [42]-[48], wherein the analysis is used to analyze extracellular vesicle subpopulations.

[50] The method of [1], wherein in said method, each of steps (a)-(c) are semi-automated using an automated liquid handler; the culturing comprises culturing said cells on a surface of a culture vessel, said culture vessel being a multi-well plate or a micro-well plate; said method further comprises blending or combining two or more culture media together, from an initial selection of at least two different culture media, to produce a panel of different candidate vesiculation culture media; said method comprises, in step (d), analyzing cell number and cell viability using an automated cell counter; said method further comprises, in step (d), measuring the total extracellular vesicle number and/or the extracellular vesicle number per cell, by measuring the expression of at least one marker present on extracellular vesicles by a high-throughput immunoassay; and said method further comprises, in step (d), analyzing marker expression and/or vesicle size of individual extracellular vesicles by super resolution microscopy.

[51] The method of [50], wherein the immunoassay is a Tim4-capture ELISA.

[52] The method of [51], wherein the ELISA is a Tim4-capture ELISA that detects CD63 expression.

[53] The method of any one of [50]-[52], wherein the analysis comprises analyzing one or more of CD9, CD63 and CD81 expression by said super resolution microscopy.

[54] The method of [53], wherein the analysis comprises analyzing CD9, CD63 and CD81 expression by said super resolution microscopy.

[55] The method of any one of [53] and [54], further comprising analyzing the size of individual extracellular vesicles by said super resolution microscopy.

[56] The method of any one of [1]-[55], further comprising selecting a candidate vesiculation media based on the results of the analysis of step (d).

[57] The method of [56], wherein the selected media provides an improvement in one or more of cell growth, cell viability, total extracellular vesicle number, extracellular vesicle number per cell, marker expression on extracellular vesicles, and extracellular vesicle size, as compared to a control benchmark medium, or an unblended medium.

[58]A vesiculation media selected by the method of [56] or [57].

[59]A system for performing the method of any one of [1]-[57], wherein said system comprises one or more of an automated liquid handler, an automated cell counter, an immunoassay kit, and a super resolution microscope.

[60] The system of [59], wherein the immunoassay kit is an ELISA kit.

[61] The method of any one of [15]-[18], wherein the panel of candidate vesiculation culture media is produced by blending or combining three or more culture media.

[62] The method of any one of [15]-[18], wherein the panel of candidate vesiculation culture media is produced by blending or combining four or more culture media.

[63] The method of any one of [50]-[55], wherein the blending or combining of the two or more culture media together is from an initial selection of at least five different culture media.

[64] The method of any one of [50]-[55], wherein the analysis by super resolution microscopy includes immobilizing extracellular vesicles on at least one of a coverslip and a microscopy channel slide.

[65] The method of any one of [50]-[55] and [64], wherein the analysis by super resolution microscopy includes detecting the at least one marker by using a fluorescent antibody.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a flowchart of the various stages of an exemplary assay described in Examples 1 and 2.

FIG. 2 depicts viable cell density after culture with the control and M1-M31 vesiculation media formulations as described in Example 1.

FIGS. 3A and 3B depict extracellular vesicle production by cells after culture in vesiculation media, measured by detecting the presence of CD63 on Tim4-captured extracellular vesicles by an extracellular vesicle ELISA (EV ELISA) assay as described in Example 1. FIG. 3A depicts total extracellular vesicle production. FIG. 3B depicts extracellular vesicle production per cell.

FIG. 4 depicts the results of a comparison between manual handling of tissue culture liquid and semi-automated handling of tissue culture liquid with respect to extracellular vesicle production (as measured by detecting the presence of CD63 on Tim4-captured extracellular vesicles by the EV ELISA assay) as described in Example 1.

FIG. 5A depicts the total extracellular vesicle counts determined from the single extracellular vesicle (dSTORM) analysis as described in Example 1.

FIG. 5B depicts the results of the nanoparticle tracking analysis (NTA). MC stands for conditioned media after the vesiculation and MV stands for virgin media control as described in Example 1.

FIG. 5C depicts CD63 expression level determined using the EV ELISA assay as described in Example 1.

FIG. 6 depicts the results of a human cardiomyocyte survival assay performed to evaluate EV function as described in Example 1.

FIG. 7 depicts the results of a HUVEC scratch wound healing assay performed to evaluate EV function as described in Example 1.

FIG. 8 depicts the viable cell density after culturing with the control and M1-M46 vesiculation media formulations (right vertical axis), and the total extracellular vesicle production as measured by the amount of CD63, CD9 and CD81 by the EV ELISA assay (left vertical axis) as described in Example 2.

FIG. 9 depicts the extracellular vesicle production per cell after culturing with the control and M1-M46 vesiculation media formulations as described in Example 2 calculated by dividing the signal from the CD63, CD9 and CD81 EV ELISA assay by viable cell count.

FIG. 10 depicts the results of the single extracellular vesicle level analysis using super resolution microscopy as described in Example 2.

FIGS. 11A and 11B show CD63, CD9 and CD81 expression level determined using the EV ELISA assay for vesicles produced with the specified vesiculation media by the manual method in flasks (FIG. 11A) and by using the automated liquid handler in multi-well plates (FIG. 11B).

FIG. 12 depicts the results of a HUVEC scratch wound healing assay performed to evaluate EV function as described in Example 2.

FIG. 13 depicts an exemplary workflow for vesiculation media development and/or optimization. In this exemplary workflow, media blending is strategized using a Design of Experiments (DoE) approach, and the cell culture liquid handling (e.g., media blending or mixing, media dispensing, media exchange, and media recovery) is performed in a semi-automated fashion using an automated liquid handler. Following culture, cell number and viability is measured using an automated cell counter. Initial screening of extracellular vesicles in conditioned media is performed using a Tim4-capture extracellular vesicle ELISA (using antibodies that bind to CD63 on extracellular vesicles as detection antibodies). Further analysis of extracellular vesicles is performed at the individual extracellular vesicle level by super resolution microscopy.

### DETAILED DESCRIPTION OF THE INVENTION

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a cell” includes one or more cells.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

As used herein, “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females. In some embodiments, cells (for example, stem cells, including pluripotent stem cells, progenitor cells, or tissue-specific cells) are derived from a subject. In some embodiments, the subject is a non-human subject.

As used herein, “differentiation” refers to processes by which unspecialized cells (such as pluripotent stem cells, or other stem cells), or multipotent or oligopotent cells, for example, acquire, are primed for, or are directed to, specialized structural and/or functional features characteristic of more mature, or fully mature, cells. “Transdifferentiation” is a process of transforming one differentiated cell type into another differentiated cell type.

As used herein, “embryoid bodies” refers to three-dimensional aggregates of pluripotent stem cells. These cells can undergo differentiation into cells of the three germ layers, the endoderm, mesoderm and ectoderm. The three-dimensional structure, including the establishment of complex cell adhesions and paracrine signaling within the embryoid body microenvironment, enables differentiation and morphogenesis.

As used herein, “stem cell” refers to a cell that has the capacity for self-renewal, i.e., the ability to go through numerous cycles of cell division while maintaining their non-terminally-differentiated state. Stem cells can be totipotent, pluripotent, multipotent, oligopotent, or unipotent. Stem cells may be, for example, embryonic, fetal, amniotic, adult, or induced pluripotent stem cells.

As used herein, “pluripotent stem cell” (PSC) refers to a cell that has the ability to reproduce itself indefinitely, and to differentiate into any other cell type of an adult organism. Generally, pluripotent stem cells are stem cells that are capable of inducing teratomas when transplanted in immunodeficient (SCID) mice; are capable of differentiating into cell types of all three germ layers (e.g., can differentiate into ectodermal, mesodermal, and endodermal, cell types); and express one or more markers characteristic of PSCs. Examples of such markers expressed by PSCs, such as embryonic stem cells (ESCs) and iPSCs, include Oct 4, alkaline phosphatase, SSEA-3 surface antigen, SSEA-4 surface antigen, nanog, TRA-1-60, TRA-1-81, SOX2, and/or REX1.

As used herein, “induced pluripotent stem cell” (iPSC) refers to a type of pluripotent stem cell that is artificially derived from a non-pluripotent cell, typically a somatic cell. In some embodiments, the somatic cell is a human somatic cell. Examples of somatic cells include, but are not limited to, dermal fibroblasts, bone marrow-derived mesenchymal cells, HPSc, hematopoietic, cardiac muscle cells, keratinocytes, liver cells, stomach cells, neural stem cells, lung cells, kidney cells, spleen cells, and pancreatic cells. Additional examples of somatic cells include cells of the immune system, including, but not limited to, B-cells, dendritic cells, granulocytes, innate lymphoid cells, megakaryocytes, monocytes/macrophages, myeloid-derived suppressor cells, natural killer (NK) cells, T cells, thymocytes, and hematopoietic stem cells. iPSCs may be generated by reprogramming a somatic cell, by expressing or inducing expression of one or a combination of factors (herein referred to as reprogramming factors) in the somatic cell. iPSCs can be generated using fetal, postnatal, newborn, juvenile, or adult somatic cells. In some instances, factors that can be used to reprogram somatic cells to pluripotent stem cells include, for example, OCT4 (OCT3/4), SOX2, c-MYC, and KLF4, NANOG, and LIN28. In some instances, somatic cells may be reprogrammed by expressing at least two reprogramming factors, at least three reprogramming factors, or at least four reprogramming factors, to reprogram a somatic cell to a pluripotent stem cell. The cells may be reprogrammed by introducing reprogramming factors using vectors, including, for example, episomal vectors, non-viral vectors, lentivirus, retrovirus, adenovirus, and Sendai virus vectors. Alternatively, non-viral techniques for introducing reprogramming factors include, for example, mRNA transfection, miRNA infection/transfection, PiggyBac, minicircle vectors, and episomal plasmids. iPSCs may also be generated by, for example, using CRISPR-Cas9-based techniques, to introduce reprogramming factors, or to activate endogenous programming genes.

As used herein, “embryonic stem cells” are embryonic cells derived from embryo tissue, preferably the inner cell mass of blastocysts or morulae, optionally that have been serially passaged as cell lines. The term includes cells isolated from one or more blastomeres of an embryo, preferably without destroying the remainder of the embryo. The term also includes cells produced by somatic cell nuclear transfer. ESCs can be produced or derived from a zygote, blastomere, or blastocyst-staged mammalian embryo produced by the fusion of a sperm and egg cell, nuclear transfer, or parthenogenesis, for example. Human ESCs include, without limitation, MAO1, MAO9, ACT-4, No. 3, H1, H7, H9, H14 and ACT30 embryonic stem cells. Exemplary pluripotent stem cells include embryonic stem cells derived from the inner cell mass (ICM) of blastocyst stage embryos, as well as embryonic stem cells derived from one or more blastomeres of a cleavage stage or morula stage embryo. These embryonic stem cells can be generated from embryonic material produced by fertilization or by asexual means, including somatic cell nuclear transfer (SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro).

As used herein, the term “progenitor cell” refers to a descendant of a stem cell which is capable of further differentiation into one or more kinds of specialized cells, but which cannot divide and reproduce indefinitely. That is, unlike stem cells (which possess an unlimited capacity for self-renewal), progenitor cells possess only a limited capacity for self-renewal. Progenitor cells may be multipotent, oligopotent, or unipotent, and are typically classified according to the types of specialized cells they can differentiate into. For instance, a “cardiomyocyte progenitor cell” is a progenitor cell derived from a stem cell that has the capacity to differentiate into a cardiomyocyte. Similarly, “cardiac progenitor cells” may differentiate into multiple specialized cells constituting cardiac tissue, including, for example, cardiomyocytes, smooth muscle cells, and endothelial cells. Additionally, a “cardiovascular progenitor cell” has the capacity to differentiate into, for example, cells of cardiac and vascular lineages.

As used herein, “expand” or “proliferate” may refer to a process by which the number of cells in a cell culture is increased due to cell division. The culture in which this expansion occurs may be known as, for example, an “expansion culture.”

“Multipotent” implies that a cell is capable, through its progeny, of giving rise to several different cell types found in an adult animal.

“Pluripotent” implies that a cell is capable, through its progeny, of giving rise to all the cell types that comprise the adult animal, including the germ cells. Embryonic stem cells, induced pluripotent stem cells, and embryonic germ cells are pluripotent cells under this definition.

The term “autologous cells” as used herein refers to donor cells that are genetically identical with the recipient.

As used herein, the term “allogeneic cells” refers to cells derived from a different, genetically non-identical, individual of the same species.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps.

As used herein, the term “extracellular vesicles” collectively refers to biological particles derived from cells, and examples thereof include exosomes, ectosomes, exovesicles, microparticles, microvesicles, nanovesicles, blebbing vesicles, budding vesicles, exosome-like vesicles, matrix vesicles, membrane vesicles, shedding vesicles, membrane particles, shedding microvesicles, oncosomes, exomeres, and/or apoptotic bodies, but are not limited thereto.

Extracellular vesicles can be categorized, for example, according to size. For instance, as used herein, the term “small extracellular vesicle” refers to extracellular vesicles having a diameter of between about 50-200 nm. In contrast, extracellular vesicles having a diameter of more than about 200 nm, but less than 400 nm, may be referred to as “medium extracellular vesicles,” and extracellular vesicles having a diameter of more than about 400 nm may be referred to as “large extracellular vesicles.” As used herein, the term “small extracellular vesicle fraction” (“sEV”) refers to a part, extract, or fraction, of secretome or conditioned medium, that is concentrated and/or enriched for small extracellular vesicles having a diameter of between about 50-200 nm. In the context of extracellular vesicle production, a cell which produces extracellular vesicles may be known as a “producer cell.”

The term “exosome” as used herein refers to an extracellular vesicle that is released from a cell upon fusion of the multivesicular body (MVB) (an intermediate endocytic compartment) with the plasma membrane.

“Exosome-like vesicles,” which have a common origin with exosomes, are typically described as having size and sedimentation properties that distinguish them from exosomes and, particularly, as lacking lipid raft microdomains. “Ectosomes,” as used herein, are typically neutrophil- or monocyte-derived microvesicles.

“Microparticles” as used herein are typically about 100-1000 nm in diameter and originate from the plasma membrane. “Extracellular membranous structures” also include linear or folded membrane fragments, e.g., from necrotic death, as well as membranous structures from other cellular sources, including secreted lysosomes and nanotubes.

As used herein, “apoptotic blebs or bodies” are typically about 1 to 5 μm in diameter and are released as blebs of cells undergoing apoptosis, i.e., diseased, unwanted and/or aberrant cells.

Within the class of extracellular vesicles, important components are “exosomes” themselves, which may be between about 40 to 50 nm and about 200 nm in diameter and being membranous vesicles, i.e., vesicles surrounded by a phospholipid bilayer, of endocytic origin, which result from exocytic fusion, or “exocytosis” of multivesicular bodies (MVBs). In some cases, exosomes can be between about 40 to 50 nm up to about 200 nm in diameter, such as being from 60 nm to 180 nm.

As used herein, the terms “secretome” and “secretome composition” interchangeably refer to one or more molecules and/or biological factors that are secreted by cells into the extracellular space (such as into a culture medium). A secretome or secretome composition may include, without limitation, extracellular vesicles (e.g., exosomes, microparticles, etc.), proteins, nucleic acids, cytokines, and/or other molecules secreted by cells into the extracellular space (such as into a culture medium). A secretome or secretome composition may be left unpurified or further processed (for example, components of a secretome or secretome composition may be present within culture medium, such as in a conditioned medium; or alternatively, components of a secretome or secretome composition may be purified, isolated, and/or enriched, from a culture medium or extract, part, or fraction thereof). A secretome or secretome composition may further comprise one or more substances that are not secreted from a cell (e.g., culture media, additives, nutrients, etc.). Alternatively, a secretome or secretome composition does not comprise one or more substances (or comprises only trace amounts thereof) that are not secreted from a cell (e.g., culture media, additives, nutrients, etc.).

As used herein, the term “conditioned medium” refers to a culture medium (or extract, part, or fraction thereof) in which one or more cells of interest have been cultured. Preferably, conditioned medium is separated from the cultured cells before use and/or further processing. The culturing of cells in culture medium may result in the secretion and/or accumulation of one or more molecules and/or biological factors (which may include, without limitation, extracellular vesicles (e.g., exosomes, microparticles, etc.), proteins, nucleic acids, cytokines, and/or other molecules secreted by cells into the extracellular space); the medium containing the one or more molecules and/or biological factors is a conditioned medium. Examples of methods of preparing conditioned media have been described in, for example, U.S. Pat. No. 6,372,494, which is incorporated by reference herein in its entirety.

As used herein, the term “cell culture” refers to cells grown under controlled condition(s) outside the natural environment of the cells. For instance, cells can be propagated completely outside of their natural environment (in vitro), or can be removed from their natural environment and then cultured (ex vivo). During cell culture, cells may survive in a non-replicative state, or may replicate and grow in number, depending on, for example, the specific culture media, the culture conditions, and the type of cells. An in vitro environment can be any medium known in the art that is suitable for maintaining cells in vitro, such as suitable liquid media or agar, for example.

The term “cell line” as used herein can refer to cultured cells that can be passaged at least one time without terminating.

The term “suspension” as used herein can refer to cell culture conditions in which cells are not attached to a solid support. Cells proliferating in suspension can be stirred while proliferating using an apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel that has been supplemented with medium suitable for further cell proliferation.

As used herein, the terms “culture medium,” “growth medium” or “medium” are used interchangeably and refer to a composition that is intended to support the growth and survival of cells. While culture media is often in liquid form, other physical forms may be used, such as, for example, a solid, semi-solid, gel, suspension, and the like. The term “vesiculation media,” as used herein, refers to a medium in which cells are cultured with the intent and/or result of producing extracellular vesicles, e.g., to produce a conditioned medium containing extracellular vesicles.

As used herein, the term “serum-free,” in the context of a culture medium or growth medium, refers to a culture or growth medium in which serum is absent. Serum typically refers to the liquid component of clotted blood, after the clotting factors (e.g., fibrinogen and prothrombin) have been removed by clot formation. Serum, such as fetal bovine serum, is routinely used in the art as a component of cell culture media, as the various proteins and growth factors therein are particularly useful for the survival, growth, and division of cells.

As used herein, the term “basal medium” refers to an unsupplemented synthetic medium that may contain buffers, one or more carbon sources, amino acids, and salts. Depending on the application, basal medium may be supplemented with growth factors and supplements, including, but not limited to, additional buffering agents, amino acids, antibiotics, proteins, and growth factors useful, for instance, for promoting growth, or maintaining or changing differentiation status, of particular cell types (e.g., fibroblast growth factor-basic (bFGF), also known as fibroblast growth factor 2 (FGF-2)).

As used herein, the terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

As used herein, the term “isolated” refers to material removed from its original environment, and is thus altered “by the hand of man” from its natural state.

As used herein, the term “enriched” means to selectively concentrate or increase the amount of one or more components in a composition, with respect to one or more other components. For instance, enrichment may include reducing or decreasing the amount of (e.g., removing or eliminating) unwanted materials; and/or may include specifically selecting or isolating desirable materials from a composition.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

As used herein, the terms “nucleic acid sequence,” “nucleotide sequence,” and “oligonucleotide” all refer to polymeric forms of nucleotides. As used herein, the term “polynucleotide” refers to a polymeric form of nucleotides that, when in linear form, has one 5′ end and one 3′ end, and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, MA) nucleosides, glycol nucleic acid, bridged nucleic acids, and morpholino structures. Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA. Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., Exonerate, BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is often between about 90% identity and 100% identity over the length of the reference polynucleotide or polypeptide or query sequence, for example, about 90% identity or higher, about 91% identity or higher, about 92% identity or higher, about 93% identity or higher, about 94% identity or higher, about 95% identity or higher, about 96% identity or higher, about 97% identity or higher, about 98% identity or higher, or about 99% identity or higher, over the length of the reference polynucleotide or polypeptide or query sequence. Sequence identity can also be calculated for the overlapping region of two sequences where only a portion of the two sequences can be aligned.

A moderate degree of sequence identity between two polynucleotides or two polypeptides is often between about 80% identity to about 90% identity over the length of the reference polynucleotide or polypeptide or query sequence, for example, about 80% identity or higher, about 81% identity or higher, about 82% identity or higher, about 83% identity or higher, about 84% identity or higher, about 85% identity or higher, about 86% identity or higher, about 87% identity or higher, about 88% identity or higher, or about 89% identity or higher, but less than 90%, over the length of the reference polynucleotide or polypeptide or query sequence.

A low degree of sequence identity between two polynucleotides or two polypeptides is often between about 50% identity and 75% identity over the length of the reference polynucleotide or polypeptide or query sequence, for example, about 50% identity or higher, about 60% identity or higher, about 70% identity or higher, but less than 75% identity, over the length of the reference polynucleotide or polypeptide or query sequence.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

“Gene” as used herein refers to a polynucleotide sequence comprising exons and related regulatory sequences. A gene may further comprise introns and/or untranslated regions (UTRs).

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, a “different” or “altered” level of, for example, a characteristic or property, is a difference that is measurably different, and preferably, statistically significant (for example, not attributable to the standard error of the assay). In some embodiments, a difference, e.g., as compared to a control or reference sample, may be, for example, a greater than 10% difference, a greater than 20% difference, a greater than 30% difference, a greater than 40% difference, a greater than 50% difference, a greater than 60% difference, a greater than 70% difference, a greater than 80% difference, a greater than 90% difference, a greater than 2-fold difference; a greater than 5-fold difference; a greater than 10-fold difference; a greater than 20-fold difference; a greater than 50-fold difference; a greater than 75-fold difference; a greater than 100-fold difference; a greater than 250-fold difference; a greater than 500-fold difference; a greater than 750-fold difference; or a greater than 1,000-fold difference, for example.

As used herein, the term “between” is inclusive of end values in a given range (e.g., between about 1 and about 50 nucleotides in length includes 1 nucleotide and 50 nucleotides).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description.

The term “effective amount,” e.g., of a composition or product, refers to a sufficient amount of the composition or product to provide the desired response.

“Transformation” as used herein refers to the insertion of an exogenous polynucleotide into a host cell, irrespective of the method used for insertion. For example, transformation can be by direct uptake, transfection, infection, and the like. The exogenous polynucleotide may be maintained as a nonintegrated vector, for example, an episome, or, alternatively, may be integrated into the host genome.

As used herein, the term “hypoxia” or “hypoxic” refers to a condition where the oxygen (O₂) concentration is below atmospheric O₂ concentration (typically 20-21%). In some embodiments, hypoxia refers to a condition with an O₂ concentration that is between 0% and 19%, between 2% and 18%, between 3% and 17%, between 4% and 16%, between 5% and 15%, between 5% and 10%, or less than 10%, less than 9%, less than 8%, less than 7%, less than 6%, less than 5%, less than 4%, less than 3%, less than 2%, or less than 1%.

As used herein, the term “normoxia” refers to a normal atmospheric concentration of oxygen, typically around 20% to 21% O₂.

As used herein, the term “high-throughput,” with respect to a process for the development, testing, and/or optimization, of culture media (and/or culture conditions) for extracellular vesicle production, refers to a process that allows for the processing of an increased number of samples and/or a decreased experiment time, and frequently both. Often, but not necessarily, a high-throughput process will employ one or more steps or assays having some level of automation (via a machine, device, computer, etc.), for example, a semi-automated or fully-automated step or assay.

As used herein, the term “automated,” in the context of a process or step, refers to a process or step in which some part(s), or no part, of the process or step is performed manually, provided that not all of the process or step is performed manually (e.g., it encompasses the concepts of semi-automated and fully-automated).

As used herein, the term “semi-automated,” in the context of a process or step, refers to a process or step in which some aspect, part, or portion, of the process or step is performed manually (such as, for example, without the aid of a liquid handler), provided that not all of the process or step is performed manually.

As used herein, the term “fully-automated,” in the context of a process or step, refers to a process or step in which no part (or substantially no part) of the process or step is performed manually.

### Extracellular Vesicle-Secreting Cells

The present disclosure relates, in part, to methods and systems for the analysis, development, testing, and/or optimization, of culture media (and/or culture conditions) for extracellular vesicle production. Cells that may be used for extracellular vesicle production in such methods and systems include, but are not limited to, stem cells, progenitor cells, and differentiated cells (including terminally- or partially-differentiated cells). Such cells may be obtained, for example, by isolation from a subject or tissue; from an in vitro cell line or culture; or via differentiation induction in vitro (e.g., progenitor cells may be generated from pluripotent stem cells, such as from embryonic stem (ES) cells or induced pluripotent stem cells (iPSCs)).

Generation of iPSC Cells

iPSC cells may be obtained from, for example, somatic cells, including human somatic cells. The somatic cell may be derived from a human or non-human animal, including, for example, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like.

In some embodiments, the somatic cell is selected from keratinizing epithelial cells, mucosal epithelial cells, exocrine gland epithelial cells, endocrine cells, liver cells, epithelial cells, endothelial cells, fibroblasts, muscle cells, cells of the blood and the immune system, cells of the nervous system including nerve cells and glial cells, pigment cells, and progenitor cells, including hematopoietic stem cells. The somatic cell may be fully differentiated (specialized), or may be less than fully differentiated. For instance, undifferentiated progenitor cells that are not PSCs, including somatic stem cells, and finally differentiated mature cells, can be used. The somatic cell may be from an animal of any age, including adult and fetal cells.

The somatic cell may be of mammalian origin. Allogeneic or autologous stem cells can be used, if for example, the secretome (or extracellular vesicles) from a progenitor cell thereof is used for administration in vivo. In some embodiments, iPSCs are not MHC-/HLA-matched to a subject. In some embodiments, iPSCs are MHC-/HLA-matched to a subject. In embodiments, for example, where iPSCs are to be used to produce PSC-derived cells, such as progenitor cells (to obtain a secretome, or extracellular vesicles), somatic cells may be obtained from the subject to be treated, or from another subject with the same or substantially the same HLA type as that of the subject. Somatic cells can be cultured before nuclear reprogramming, or can be reprogrammed without culturing after isolation, for example.

To introduce reprogramming factors into somatic cells, for example, viral vectors may be used, including, e.g., vectors from viruses such as SV40, adenovirus, vaccinia virus, adeno-associated virus, herpes viruses including HSV and EBV, Sindbis viruses, alphaviruses, human herpesvirus vectors (HHV) such as HHV-6 and HHV-7, and retroviruses. Lentiviruses include, but are not limited to, Human Immunodeficiency Virus type 1 (HIV-1), Human Immunodeficiency Virus type 2 (HIV-2), Simian Immunodeficiency Virus (SIV), Feline Immunodeficiency Virus (FIV), Equine Infectious Anaemia Virus (EIAV), Bovine Immunodeficiency Virus (BIV), Visna Virus of sheep (VISNA) and Caprine Arthritis-Encephalitis Virus (CAEV). Lentiviral vectors are capable of infecting non-dividing cells and can be used for both in vivo and in vitro gene transfer and expression of nucleic acid sequences. A viral vector can be targeted to a specific cell type by linkage of a viral protein, such as an envelope protein, to a binding agent, such as an antibody, or a particular ligand (for targeting to, for instance, a receptor or protein on or within a particular cell type).

In some embodiments, a viral vector, such as a lentiviral vector, can integrate into the genome of the host cell. The genetic material thus transferred is then transcribed and possibly translated into proteins inside the host cell. In other embodiments, viral vectors are used that do not integrate into the genome of a host cell.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector, for example.

Somatic cells can be reprogrammed to produce induced pluripotent stem cells (iPSCs) using methods known to one of skill in the art. One of skill in the art can readily produce induced pluripotent stem cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; and 8,268,620, all of which are incorporated herein by reference.

Generally, reprogramming factors which can be used to create induced pluripotent stem cells, either singly, in combination, or as fusions with transactivation domains, include, but are not limited to, one or more of the following genes: Oct4 (Oct3/4, Pou5f1), Sox (e.g., Sox1, Sox2, Sox3, Sox18, or Sox15), Klf (e.g., Klf4, Klf1, Klf3, Klf2 or Klf5), Myc (e.g., c-myc, N-myc or L-myc), nanog, or LIN28. As examples of sequences for these genes and proteins, the following accession numbers are provided: Mouse MyoD: M84918, NM_010866; Mouse Oct4 (POU5F1): NM_013633; Mouse Sox2: NM_011443; Mouse Klf4: NM_010637; Mouse c-Myc: NM_001177352, NM_001177353, NM 001177354 Mouse Nanog: NM 028016; Mouse Lin28: NM 145833: Human MyoD: NM_002478; Human Oct4 (POU5F1): NM_002701, NM 203289, NM_001173531; Human Sox2: NM_003106; Human Klf4: NM_004235; Human c-Myc: NM_002467; Human Nanog: NM_024865; and/or Human Lin28: NM_024674. Also contemplated are sequences similar thereto, including those having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity. In some embodiments, at least three, or at least four, of Klf4, c-Myc, Oct3/4, Sox2, Nanog, and Lin28 are utilized. In other embodiments, Oct3/4, Sox2, c-Myc and Klf4 are utilized.

Exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV40LT); (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmi1; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV40LT; (14) Oct3/4, Klf4, Sox2, TERT, HPV16 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmi1; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg).

iPSCs typically display the characteristic morphology of human embryonic stem cells (hESCs), and express the pluripotency factor, NANOG. Embryonic stem cell specific surface antigens (SSEA-3, SSEA-4, TRA1-60, TRA1-81) may also be used to identify fully reprogrammed human cells. Additionally, at a functional level, PSCs, such as ESCs and iPSCs, also demonstrate the ability to differentiate into lineages from all three embryonic germ layers, and form teratomas in vivo (e.g., in SCID mice).

### Differentiation of PSCs

The present disclosure further contemplates differentiating PSCs, including ESCs and iPSCs, to produce extracellular vesicle-producing cells having a more differentiated state than PSCs. For instance, PSCs can be differentiated into terminally-differentiated (specialized) cells that can be used to produce extracellular vesicles; or differentiated into progenitor cells that can be used to produce extracellular vesicles.

Progenitor cells of the present disclosure include, for example, hematopoietic progenitor cells, myeloid progenitor cells, neural progenitor cells; pancreatic progenitor cells, cardiac progenitor cells, cardiomyocyte progenitor cells, cardiovascular progenitor cells, renal progenitor cells, skeletal myoblasts, satellite cells, intermediate progenitor cells formed in the subventricular zone, radial glial cells, bone marrow stromal cells, periosteum cells, endothelial progenitor cells, blast cells, boundary cap cells, and mesenchymal stem cells. Methods for differentiating pluripotent stem cells to progenitor cells, and for culturing and maintaining progenitor cells, are known in the art, such as those described in U.S. Provisional Patent Application No. 63/243,606 entitled “Methods for the Production of Committed Cardiac Progenitor Cells,” which is incorporated by reference herein in its entirety.

Specialized cells of the present disclosure include, for example, fibroblasts, muscle cells, keratinocytes, liver cells, stomach cells, neural cells, lung cells, kidney cells, spleen cells, endothelial cells, and pancreatic cells; as well as cells of the immune system, including, but not limited to, B-cells, dendritic cells, granulocytes, innate lymphoid cells, megakaryocytes, monocytes/macrophages, myeloid-derived suppressor cells, natural killer (NK) cells, and T cells.

Analysis, Development, Testing, and/or Optimization, of Culture Media and/or Culture Conditions for Extracellular Vesicle Production

The present disclosure provides methods and systems for the analysis, development, testing, and/or optimization, of culture media (and/or culture conditions) for extracellular vesicle production. For example, in some embodiments, the present disclosure provides high-throughput methods for one or more of cell culture; liquid handling (including, e.g., media dispensing, media exchange, and media harvesting); vesiculation; analysis of cell growth and/or viability; analysis of extracellular vesicle production and secretion; and characterization of extracellular vesicles, thereby providing improved methods and systems for the analysis, development, testing, and/or optimization, of culture media (and/or culture conditions) for extracellular vesicle production (such as by allowing an increase in the number of test samples, and/or a reduction in the length of time).

In some embodiments, one or more types of extracellular vesicle-producing cells (i.e., cells from which extracellular vesicles may be obtained, also termed producer cells) are initially subjected to one or more culture expansion steps before a vesiculation step.

In some embodiments, at least 1, at least 2, at least 3, at least 4, at least 5, at least 6, at least 7, at least 8, at least 9, at least 10, at least 15, or at least 20, different types of producer cells are cultured in parallel, and subsequently used for extracellular vesicle production.

The one or more producer cells can be, for example, cells that have recently been isolated or differentiated (e.g., from stem cells). Alternatively, in some embodiments, cells that have previously been refrigerated, frozen, and/or cryopreserved, may be used in the culturing methods. In some embodiments, cells are thawed from a cryopreserved state (e.g., −80° C. or colder) before use. In some embodiments thereof, the cells are thawed in a thawing medium. In some embodiments, the thawing medium may comprise a liquid medium containing one or more supplements. In some embodiments, the cells may be thawed in a thawing device, such as, for example, a water bath or a water-free thawing system (e.g., ThawSTAR™ Automated Thawing System, Biolife Solutions®).

Each type of producer cell cultured is preferably, although not necessarily, cultured in multiplicate. For example, each type of producer cell may be cultured in duplicate, triplicate, quadruplicate, quintuplicate, sextuplicate, septuplicate, octuplicate, etc., depending on the number of vesiculation media preparations, and/or culture conditions, etc., to be analyzed. In some embodiments, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 500, at least 1000, at least 5000, at least 10,000, at least 50,000, or at least 100,000, producer cell cultures (of the same or different cells) are cultured in parallel. The cell culturing may be adherent or non-adherent (e.g., suspension) cell culture. The culturing may be two-dimensional or three-dimensional cell culturing.

In some embodiments, the culture vessel used for culturing may be a flask, flask for tissue culture (e.g., T25, T75), hyperflask (e.g., CellBind surface HYPERFlask®; Corning, Ref: 10024) or hyperstack (e.g., 12 or 36 chamber, HYPERStacks®, Corning, Refs: 10012, 10036, 10013, 10037), dish, petri dish, dish for tissue culture, multi dish, micro plate, micro-well plate, multi plate, multi-well plate, micro slide, chamber slide, tube, tray, CellSTACK® Chambers (e.g., 1ST, 2ST, 5ST, 10ST; Corning, Refs: 3268, 3269, 3313, 3319), culture bag, roller bottle, bioreactor, stirred culture vessel, spinner flask, microcarrier, or a vertical wheel bioreactor, for example. The cells may be cultured in a volume of at least or about 0.2, 0.5, 1, 2, 5, 10, 15, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, 1 L, 5 L, 10 L, 50 L, 100 L, 1000 L, 5000 L, or 10,000 L, for example.

In some embodiments, multiplicate cell cultures are preferably cultured within different compartments or wells of a tissue culture vessel. For instance, in some embodiments, multiplicate cell cultures are adherent-cultured within different wells or portions of a tissue culture vessel, such as a micro-well plate or a multi-well plate (e.g., a plate containing at least 6 wells, at least 12 wells, at least 24 wells, at least 48 wells, at least 96 wells, at least 128 wells, at least 256 wells, at least 384 wells, at least 500 wells, at least 1000 wells, at 1500 wells, at least 1536 wells, at least 2000 wells, at least 5000 wells, or at least 10,000 wells).

In embodiments in which culturing comprises two-dimensional cell culture, such as on the surface of a culture vessel, the culture surface (to which the cells are intended to adhere) may be coated with one or more substances that promote cell adhesion. Such substances useful for enhancing attachment to a solid support include, for example, type I, type II, and type IV collagen, concanavalin A, chondroitin sulfate, fibronectin, fibronectin-like polymers, gelatin, laminin, poly-D and poly-L-lysine, Matrigel, thrombospondin, osteopontin, poly-D-lysine, human extracellular matrix, Corning® Cell-Tak™ Cell and Tissue Adhesive, Corning PuraMatrix® Peptide Hydrogel, and/or vitronectin.

In some embodiments, where culturing of cells is performed as adherent culture, e.g., where cells are adhered to a solid support, cells may be seeded at an amount of 25,000-250,000 cells per cm²; 50,000-200,000 cells per cm²; 75,000-175,000 cells per cm²; or between 100,000-150,000 cells per cm². In some embodiments, where culturing of cells is performed as adherent culture, cells may be seeded to the solid support under gravitational force. In other embodiments, the cells may be seeded to the solid support under centrifugation.

In some embodiments, the seeding and expansion culture of multiplicate cell cultures is preferably at least partially automated and/or is high-throughput. In some embodiments, the seeding and culturing of the multiple producer cell cultures is semi-automated. In some embodiments, the seeding and culturing of the multiple producer cell cultures is fully-automated.

The expansion culturing may be performed for differing lengths of time. For instance, the expansion culturing may be performed for a period of 6-96 hours, 12-72 hours, 36-60 hours, 42-56 hours, or for about or at least 12 hours, about or at least 18 hours, about or at least 24 hours, about or at least 30 hours, about or at least 36 hours, about or at least 42 hours, about or at least 48 hours, about or at least 54 hours, about or at least 60 hours, about or at least 66 hours, about or at least 72 hours, about or at least 78 hours, about or at least 84 hours, about or at least 90 hours, about or at least 96 hours, about or at least 120 hours, about or at least 144 hours, about or at least 168 hours, about or at least 192 hours, about or at least 1 week, about or at least 2 weeks, about or at least 3 weeks, or about or at least 4 weeks.

In some embodiments, all or a part of the expansion culturing is performed under hypoxic conditions. In some embodiments, the hypoxic condition is an O₂ concentration that is between 0% and 15%, between 0% and 10%, or less than 10%, less than 9%, less than 8%, less than 7%, less than 6%, less than 5%, less than 4%, less than 3%, less than 2%, or less than 1%.

In some embodiments, all or a part of the expansion culturing step is performed under normoxic conditions. In some embodiments, at least the last 6-72 hours, the last 10-48 hours, or the last 12-36 hours, of the culturing step is performed under normoxic conditions. In some embodiments, the normoxic condition is an O₂ concentration that is between 20% and 21%.

Any culture medium suitable for culturing the producer cell(s) may be used, including, for example, known and commercially available cell culture mediums. In some embodiments, a basal medium containing a buffer(s), one or more carbon sources, amino acids, and salts, may be used. In some embodiments, a basal medium may be supplemented with growth factors and supplements, including, but not limited to, additional buffering agents, amino acids, antibiotics, proteins, and growth factors useful, for instance, for promoting growth, or maintaining or changing differentiation status, of particular cell types (e.g., fibroblast growth factor-basic (bFGF), also known as fibroblast growth factor 2 (FGF-2)). The basal medium may be any basal culture medium suitable for the cell type to be cultured, including, for example, culture media containing, consisting of, or comprising, Dulbecco's Modified Eagle's Medium (DMEM), DMEM F12 medium, Eagle's Minimum Essential Medium (MEM), α-MEM, F-12K medium, Iscove's Modified Dulbecco's Medium (IMDM), Knockout DMEM, RPMI-1640 medium, F-10 medium, Glasgow Modified Essential Medium (GMEM), McCoy's 5A medium, Basal Medium Eagle (BME), Medium 199, or variants, combinations, or modifications thereof.

Additional supplements can also be added to the medium to supply the cells with trace elements for optimal growth and expansion. Such supplements include, for example, insulin, transferrin, sodium selenium, Hanks' Balanced Salt Solution, Earle's Salt Solution, antioxidant supplements, MCDB-201, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids, and combinations thereof. Such amino acids include, but are not limited to, L-alanine, L-arginine, L-aspartic acid, L-asparagine, L-cysteine, L-cysteine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

Optionally, hormones can also be used in cell culture and include, but are not limited to, D-aldosterone, diethylstilbestrol (DES), dexamethasone, beta-estradiol, hydrocortisone, insulin, prolactin, progesterone, somatostatin/human growth hormone (HGH), thyrotropin, thyroxine, and L-thyronine. Beta-mercaptoethanol can also be supplemented in cell culture media.

Lipids and lipid carriers can also be used to supplement cell culture media, depending on the type of cell. Such lipids and carriers can include, but are not limited to, cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In certain embodiments, an albumin, such as human serum albumin, is present in the culture medium. The albumin, including human serum albumin, may be, for example, isolated, synthetic, recombinant, and/or modified. The amount of albumin may be adjusted depending on the desired culture conditions and/or need. In some embodiments, the albumin may be present in an amount from 0.1 μg/mL-50 mg/mL, in an amount from 1 μg/mL-25 mg/mL, in an amount from 10 μg/mL-20 mg/mL, in an amount from 100 μg/mL-10 mg/mL, in an amount from 0.5 mg/mL-5 mg/mL, in an amount from 1 mg/mL-3 mg/mL, or in an amount of about 0.5 mg/mL, 1 mg/mL, 2 mg/mL, 3 mg/mL, 4 mg/mL or 5 mg/mL.

In some embodiments, the media further comprises one or more selected from the group consisting of: glutamine; biotin; DL alpha tocopherol acetate; DL alpha-tocopherol; vitamin A; catalase; insulin; transferrin; superoxide dismutase; corticosterone; D-galactose; ethanolamine, glutathione; L-carnitine; linoleic acid; progesterone; putrescine; sodium selenite; triodo-I-thyronine; an amino acid; sodium pyruvate; lipoic acid; vitamin B12; nucleosides; and ascorbic acid. The medium may also be supplemented with one or more carbon sources. The one or more carbon sources may be selected from, for example, carbon sources such as glycerol, glucose, galactose, sucrose, fructose, mannose, lactose, or maltose. The medium may contain serum, such as fetal calf serum or fetal bovine serum, or it may be a serum-free medium.

Following expansion culture of producer cells, the culture medium used for the expansion cell culture is preferably removed and replaced with vesiculation media; after culturing the producer cells in the vesiculation media, conditioned media is thereby produced. Preferably, the conditioned media contains extracellular vesicles.

In some embodiments in which the expanded producer cells are to be used for the analysis, development, testing, and/or optimization of vesiculation media, multiple different vesiculation media formulations may be tested in parallel (on multiplicate producer cell cultures). In some embodiments, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 500, at least 1000, at least 5000, at least 10,000, at least 50,000, or at least 100,000, different vesiculation media formulations may be tested in parallel, or sequentially.

A plurality of vesiculation media formulations can be produced, for example, by blending or combining two or more different types of culture media together. In some embodiments, one or more of the culture media for blending or combining are known in the art and/or are commercially available. In some embodiments, at least 1, at least 2, at least 3, at least 4, at least 5, at least 6, at least 7, at least 8, at least 9, at least 10, at least 15, at least 20, or at least 25 or more, culture media are blended or combined together to create a particular vesiculation media formulation. The culture media for blending may be any culture media suitable for the cell type to be cultured, including, for example, culture media containing, consisting of, or comprising, Dulbecco's Modified Eagle's Medium (DMEM), DMEM F12 medium, Eagle's Minimum Essential Medium (MEM), α-MEM, F-12K medium, Iscove's Modified Dulbecco's Medium (IMDM), Knockout DMEM, RPMI-1640 medium, F-10 medium, Glasgow Modified Essential Medium (GMEM), McCoy's 5A medium, Basal Medium Eagle (BME), Medium 199, or variants, combinations, or modifications thereof.

The culture media for blending may contain one or more supplements or additives. In some embodiments, the supplement or additive may be one or more growth factors. In some embodiments, the one or more growth factors may be selected from Adrenomedullin, Angiopoietin, Autocrine motility factor, Bone morphogenetic proteins (BMPs), Ciliary neurotrophic factor (CNTF), Leukemia inhibitory factor (LIF), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF-1), Fibroblast growth factor 2 (FGF-2), Fibroblast growth factor 3 (FGF-3), Fibroblast growth factor 4 (FGF-4), Fibroblast growth factor 5 (FGF-5), Fibroblast growth factor 6 (FGF-6), Fibroblast growth factor 7 (FGF-7), Fibroblast growth factor 8 (FGF-8), Fibroblast growth factor 9 (FGF-9), Fibroblast growth factor 10 (FGF-10), Fibroblast growth factor 11 (FGF-11), Fibroblast growth factor 12 (FGF-12), Fibroblast growth factor 13 (FGF-13), Fibroblast growth factor 14 (FGF-14), Fibroblast growth factor 15 (FGF-15), Fibroblast growth factor 16 (FGF-16), Fibroblast growth factor 17 (FGF-17), Fibroblast growth factor 18 (FGF-18), Fibroblast growth factor 19 (FGF-19), Fibroblast growth factor 20 (FGF-20), Fibroblast growth factor 21 (FGF-21), Fibroblast growth factor 22 (FG-F22), Fibroblast growth factor 23 (FGF-23), Fetal Bovine Somatotrophin (FBS), Glial cell line-derived neurotrophic factor (GDNF), Neurturin, Persephin, Artemin, Growth differentiation factor-9 (GDF-9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

In some embodiments, the one or more growth factors may each independently be present in an amount from 0.001 μg/mL-1000 μg/mL, in an amount from 0.01 μg/mL-100 μg/mL, in an amount from 0.1 μg/mL-10 μg/mL, in an amount from 0.05 μg/mL-5 μg/mL, in an amount from 0.5 μg/mL-2.5 μg/mL, or in an amount of about 0.5 μg/mL, about 1 μg/mL, about 2 μg/mL, about 3 μg/mL, about 4 μg/mL or about 5 μg/mL.

Other supplements or additives may include, for example, carbon sources (such as glycerol, glucose, galactose, sucrose, fructose, mannose, lactose, or maltose), albumin, biotin, DL alpha tocopherol acetate, DL alpha-tocopherol, vitamin A, catalase, superoxide dismutase, corticosterone, D-galactose, ethanolamine, glutathione, L-carnitine, putrescine, sodium selenite, triodo-I-thyronine, sodium pyruvate, lipoic acid, vitamin B12, nucleosides, beta-mercaptoethanol, insulin, transferrin, sodium selenium, Hanks' Balanced Salt Solution, Earle's Salt Solution, antioxidant supplements, MCDB-201, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids, and combinations thereof. Such amino acids include, but are not limited to, L-alanine, L-arginine, L-aspartic acid, L-asparagine, L-cysteine, L-cysteine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine. Optionally, hormones can be added and include, but are not limited to, D-aldosterone, diethylstilbestrol (DES), dexamethasone, beta-estradiol, hydrocortisone, insulin, prolactin, progesterone, somatostatin/human growth hormone (HGH), thyrotropin, thyroxine, and L-thyronine.

Lipids and lipid carriers can also be used as additives or supplements. Such lipids and carriers can include, but are not limited to, cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In some embodiments, a panel of vesiculation media formulations is produced by blending or combining two or more different types of culture media together, from an initial selection of different culture media. In some embodiments, the initial selection of different culture media comprises at least 2, at least 3, at least 4, at least 5, at least 6, at least 7, at least 8, at least 9, at least 10, at least 20, at least 30, at least 40, at least 50, at least 75, or at least 100, different culture media.

In some embodiments the initial selection of the different culture media is performed based on suitability of a particular culture medium for a specific type of extracellular vesicle-secreting cells, culture conditions, desired results, and other considerations. In some embodiments, the initial selection of the different culture media is performed by screening different culture media for desired characteristics, for example, by culturing extracellular vesicle-secreting cells in said culture media to obtain conditioned media and analyzing one or more properties of the extracellular vesicles in the recovered conditioned media, the recovered cells or both.

In some embodiments, a panel of vesiculation media formulations is produced by blending or combining two or more different types of culture media together without any initial selection or screening of the different culture media.

Alternatively, multiple vesiculation media formulations may be generated by adding one or more supplements, additives, etc., to an existing culture medium (or to a blend of different types of culture media, as described above), e.g., in different combinations and/or at different concentrations.

In some embodiments, the blending or combining of different media; and/or the addition of one or more additives or supplements, to prepare vesiculation media formulations, may be facilitated or strategized using one or more statistical methods. In some embodiments, a Design of Experiments (DoE) approach is used. In some embodiments, the blending or combining of different media; and/or the addition of one or more additives or supplements, to prepare vesiculation media formulations, may be facilitated or strategized using software (e.g., Design Expert software, by StatEase).

In some embodiments, the supplement or additive may be one or more growth factors. In some embodiments, the one or more growth factors may be selected from Adrenomedullin, Angiopoietin, Autocrine motility factor, Bone morphogenetic proteins (BMPs), Ciliary neurotrophic factor (CNTF), Leukemia inhibitory factor (LIF), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor 1 (FGF-1), Fibroblast growth factor 2 (FGF-2), Fibroblast growth factor 3 (FGF-3), Fibroblast growth factor 4 (FGF-4), Fibroblast growth factor 5 (FGF-5), Fibroblast growth factor 6 (FGF-6), Fibroblast growth factor 7 (FGF-7), Fibroblast growth factor 8 (FGF-8), Fibroblast growth factor 9 (FGF-9), Fibroblast growth factor 10 (FGF-10), Fibroblast growth factor 11 (FGF-11), Fibroblast growth factor 12 (FGF-12), Fibroblast growth factor 13 (FGF-13), Fibroblast growth factor 14 (FGF-14), Fibroblast growth factor 15 (FGF-15), Fibroblast growth factor 16 (FGF-16), Fibroblast growth factor 17 (FGF-17), Fibroblast growth factor 18 (FGF-18), Fibroblast growth factor 19 (FGF-19), Fibroblast growth factor 20 (FGF-20), Fibroblast growth factor 21 (FGF-21), Fibroblast growth factor 22 (FG-F22), Fibroblast growth factor 23 (FGF-23), Fetal Bovine Somatotrophin (FBS), Glial cell line-derived neurotrophic factor (GDNF), Neurturin, Persephin, Artemin, Growth differentiation factor-9 (GDF-9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

In some embodiments, the one or more growth factors may each independently be present in an amount from 0.001 μg/mL-1000 μg/mL, in an amount from 0.01 μg/mL-100 μg/mL, in an amount from 0.1 μg/mL-10 μg/mL, in an amount from 0.05 μg/mL-5 μg/mL, in an amount from 0.5 μg/mL-2.5 μg/mL, or in an amount of about 0.5 μg/mL, about 1 μg/mL, about 2 μg/mL, about 3 μg/mL, about 4 μg/mL or about 5 μg/mL.

Other supplements or additives may include, for example, carbon sources (such as glycerol, glucose, galactose, sucrose, fructose, mannose, lactose, or maltose), albumin, biotin, DL alpha tocopherol acetate, DL alpha-tocopherol, vitamin A, catalase, superoxide dismutase, corticosterone, D-galactose, ethanolamine, glutathione, L-carnitine, putrescine, sodium selenite, triodo-I-thyronine, sodium pyruvate, lipoic acid, vitamin B12, nucleosides, beta-mercaptoethanol, insulin, transferrin, sodium selenium, Hanks' Balanced Salt Solution, Earle's Salt Solution, antioxidant supplements, MCDB-201, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids, and combinations thereof. Such amino acids include, but are not limited to, L-alanine, L-arginine, L-aspartic acid, L-asparagine, L-cysteine, L-cysteine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine. Optionally, hormones can be added and include, but are not limited to, D-aldosterone, diethylstilbestrol (DES), dexamethasone, beta-estradiol, hydrocortisone, insulin, prolactin, progesterone, somatostatin/human growth hormone (HGH), thyrotropin, thyroxine, and L-thyronine.

Lipids and lipid carriers can also be used as additives or supplements. Such lipids and carriers can include, but are not limited to, cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

The vesiculation culture may be performed for differing lengths of time. For instance, the culturing may be performed for a period of 6-96 hours, 12-72 hours, 36-60 hours, 42-56 hours, or for about or at least 6 hours, about or at least 12 hours, about or at least 18 hours, about or at least 24 hours, about or at least 36 hours, about or at least 48 hours, about or at least 60 hours, about or at least 72 hours, about or at least 84 hours, about or at least 96 hours, about or at least 120 hours, about or at least 144 hours, about or at least 168 hours, about or at least 192 hours, about or at least 1 week, about or at least 2 weeks, about or at least 3 weeks, or about or at least 4 weeks.

In some embodiments, all or a part of the vesiculation culturing is performed under hypoxic conditions. In some embodiments, the last 6-72 hours, the last 10-48 hours, or the last 12-36 hours, of the culturing is performed under hypoxic conditions. In some embodiments, the hypoxic condition is an O₂ concentration that is between 0% and 15%, between 0% and 10%, or less than 10%, less than 9%, less than 8%, less than 7%, less than 6%, less than 5%, less than 4%, less than 3%, less than 2%, or less than 1%.

In some embodiments, all or a part of the vesiculation culturing step is performed under normoxic conditions. In some embodiments, at least the last 6-72 hours, the last 10-48 hours, or the last 12-36 hours, of the culturing step is performed under normoxic conditions. In some embodiments, the normoxic condition is an O₂ concentration that is between 20% and 21%.

In some embodiments, removal of the expansion culture medium from the expansion cell culture(s); washing of cell cultures; formulation of vesiculation media formulation(s); addition of vesiculation media formulation(s) to cell cultures; culturing of cell cultures (expansion and/or vesiculation cultures); and/or harvesting of conditioned media(s), is at least partially automated (e.g., semi-automated or fully-automated), and/or is high-throughput. For instance, an automated liquid handler, such as a Biomek® automated liquid handler manufactured by Beckman Coulter, may be used.

During and/or after culturing of the producer cells in vesiculation media (the vesiculation stage during which conditioned media containing extracellular vesicles is produced), one or more properties of the cultured cells may be examined (including, for example: the total number of cells, cell density, the number of viable cells, the percentage viability of the cells; morphologies of the cells; identity of the cells; karyotype of the cells; transcriptome of the cells, hypertrophy, cell health, cell adhesion, cell physiology, and/or ATP content).

In some embodiments, the cultured producer cells are analyzed by counting (e.g., by determining cell density) and/or using a cell viability assay. In some embodiments, during and/or after culturing of the producer cells in vesiculation media, cells are stained with a DNA-labeling dye and/or a nuclear-staining dye and counted (e.g., using a cell counter). In some embodiments thereof, the DNA-labeling dye or the nuclear-staining dye is a fluorescent dye.

In some embodiments, during and/or after culturing of the producer cells in vesiculation media, the producer cells are stained with acridine orange and propidium iodide, and counted using a cell counter. In some embodiments, the counting of the viable and/or non-viable cells is at least partially automated (e.g., semi-automated or fully-automated) and/or is high-throughput. For instance, a Cellaca Cell Counter, manufactured by Nexcelom, may be used.

Additionally, or alternatively, one or more properties of extracellular vesicles in the conditioned media (produced by the vesiculation culture) can be analyzed using one or more assays (including, e.g., particle number; particle concentration; particle size distribution; protein concentration; protein profile concentration; RNA profile; potency; marker expression; host cell protein assessment; residual DNA quantification and/or characterization; appearance; pH; osmolarity; etc.), to determine one or more properties of the extracellular vesicles.

In some embodiments, conditioned media is analyzed to estimate or determine the number and/or types of extracellular vesicles in the conditioned media. In some embodiments, the number and/or types of extracellular vesicles in the conditioned media is estimated or determined using an affinity-based assay, such as an immunoassay. In some embodiments, the immunoassay may be a high-throughput immunoassay. In some embodiments, the immunoassay may be, for example, an enzyme-linked immunosorbent assay (ELISA), a competitive binding assay, an immunometric assay, a radioimmunoassay (RIA), a fluoroimmunoassay (FIA), a chemiluminescent immunoassay (CLIA), a counting immunoassay (CIA), or flow cytometry or FACS.

In some embodiments, the extracellular vesicles in the conditioned medium are captured using a reagent (such as a protein) having an affinity for extracellular vesicles. In some embodiments, the reagent is a Tim4 protein (or variant or derivative thereof), which has affinity for phosphatidylserine displayed on the surface of extracellular vesicles. In some embodiments, the captured extracellular vesicles can then be detected using a different reagent that binds to or detects extracellular vesicles, such as, for example, a reagent that binds to or detects any one or more markers present on extracellular vesicles. Such markers may be any marker(s) present on an extracellular vesicle. Such markers may be selected from, for example, tetraspanins (e.g., CD9, CD63 and CD81), ceramide, MHC class I, MHC class II, integrins, adhesion molecules, phosphatidylserine, sphingomyelin, cholesterol, cytoskeletal proteins (e.g., actin, gelsolin, myosin, tubulin), enzymes (e.g., catalase, GAPDH, nitric oxide synthase, LT synthases), nucleic acids (e.g., RNA, miRNA), heat shock proteins (e.g., HSC70, HSP60, HSP70, HSPA5, CCT2, and HSP90), exosome biogenesis proteins (ALIX, Tsg101), LT, prostaglandins, and S100 proteins. Other exemplary extracellular vesicle markers that may be analyzed and/or detected include, for example, one or more of CD3, flotillins (e.g., flotillin-1, flotillin-2), TSG101 (tumor susceptibility 101), CD4, CD19, CD8, HLA-DRDPDQ, CD56, CD105, CD2, CD1c, CD25, CD49e, ROR1 (Neurotrophic Tyrosine Kinase, receptor-related 1), CD209, SSEA-4 (Stage-Specific Embryonic Antigen-4), HLA-ABCG, CD40, CD62P, CD11c, MCSP (Melanoma-associated Chondroitin Sulphate Proteoglycan), CD146, CD41b, CD42a, CD24, CD86, CD44, CD326, CD133/1, CD29, CD69, CD142, CD45, CD31, CD20, CD14, Rab-5b, TSG101, annexins (e.g, annexin 2, annexin 5), programmed cell death 6 interacting protein (Alix), fibronectin 1, galectin 3 binding protein, alpha-2-macroglobulin, hemoglobin subunit beta, gelsolin, beta-actin, beta-2-microglobulin, stomatin, moesin, peroxiredoxin 2, RAP1B (member of RAS oncogene family), filamin A, integrins, selectins, syntenins (e.g., syntenin-1), Syndecan binding protein (SDCBP), 14-3-3 protein, IMMT (mitochondrial protein), Disco-interacting protein 2 homolog B (DIP2B), members of the 4-transmembrane protein family TSPAN6 and TSPAN3, arrestin domain-containing protein 1 (ARRDC1), immunoglobulin superfamily member 8 (IGSF8), CD82, TfR2, LAMP1/2, heparan sulphate proteoglycans, EMMPRIN, ADAM10, NT5E, complement-binding proteins (e.g., CD55 and CD59) glycophorin A, AChE-E, amyloid beta A4/APP, ESCRT-I/II/III, caveolins.

In some embodiments, the reagent that binds to or detects extracellular vesicles is an antibody. In some embodiments, the reagent, such as an antibody, binds to or detects CD9, CD63 or CD81. In some embodiments, the reagent is an antibody which binds CD63.

In some embodiments, the seeding and expansion culture of the multiplicate cell cultures is preferably at least partially automated and/or is high throughput. In some embodiments, the seeding and culturing of the multiplicate cell cultures is semi-automated. In some embodiments, the seeding and culturing of the multiplicate cell cultures is fully-automated.

In some embodiments, the analysis of the conditioned media to estimate or determine the number and/or types of extracellular vesicles therein is performed using a high-throughput ELISA, using Tim4 protein (or a variant or derivative thereof) to capture extracellular vesicles, and using an anti-CD63 antibody to detect captured extracellular vesicles.

In some embodiments, extracellular vesicles in conditioned media are characterized at the single extracellular vesicle level. In some embodiments, single extracellular vesicles are analyzed for the presence of one or more markers, and/or to determine their size. In some embodiments, single extracellular vesicles are analyzed using a microscopy technique. In some embodiments, the microscopy technique is a super resolution microscopy technique. In some embodiments, the super resolution microscopy technique is Direct Stochastic Optical Reconstruction Microscopy (dSTORM).

In some embodiments, extracellular vesicles are analyzed, at the single extracellular vesicle level, for the presence of one or more markers present on extracellular vesicles. Such markers may be any marker(s) present on an extracellular vesicle. Such markers may be, for example, selected from tetraspanins (e.g., CD9, CD63 and CD81), ceramide, MHC class I, MHC class II, integrins, adhesion molecules, phosphatidylserine, sphingomyelin, cholesterol, cytoskeletal proteins (e.g., actin, gelsolin, myosin, tubulin), enzymes (e.g., catalase, GAPDH, nitric oxide synthase, LT synthases), nucleic acids (e.g., RNA, miRNA), heat shock proteins (e.g., HSC70, HSP60, HSP70, HSPA5, CCT2, and HSP90), exosome biogenesis proteins (ALIX, Tsg101), LT, prostaglandins, and S100 proteins. Other exemplary extracellular vesicle markers that may be analyzed and/or detected include, for example, one or more of CD3, flotillins (e.g., flotillin-1, flotillin-2), TSG101 (tumor susceptibility 101), CD4, CD19, CD8, HLA-DRDPDQ, CD56, CD105, CD2, CD1c, CD25, CD49e, ROR1 (Neurotrophic Tyrosine Kinase, receptor-related 1), CD209, SSEA-4 (Stage-Specific Embryonic Antigen-4), HLA-ABCG, CD40, CD62P, CD11c, MCSP (Melanoma-associated Chondroitin Sulphate Proteoglycan), CD146, CD41b, CD42a, CD24, CD86, CD44, CD326, CD133/1, CD29, CD69, CD142, CD45, CD31, CD20, CD14, Rab-5b, TSG101, annexins (e.g, annexin 2, annexin 5), programmed cell death 6 interacting protein (Alix), fibronection 1, galectin 3 binding protein, alpha-2-macroglobulin, hemoglobin subunit beta, gelsolin, beta-actin, beta-2-microglobulin, stomatin, moesin, peroxiredoxin 2, RAP1B (member of RAS oncogene family), filamin A, integrins, selectins, syntenins (e.g., syntenin-1), Syndecan binding protein (SDCBP), 14-3-3 protein, IMMT (mitochondrial protein), Disco-interacting protein 2 homolog B (DIP2B), members of the 4-transmembrane protein family TSPAN6 and TSPAN3, arrestin domain-containing protein 1 (ARRDC1), immunoglobulin superfamily member 8 (IGSF8), CD82, TfR2, LAMP1/2, heparan sulphate proteoglycans, EMMPRIN, ADAM10, NT5E, complement-binding proteins (e.g., CD55 and CD59) glycophorin A, AChE-E, amyloid beta A4/APP, ESCRT-I/II/III, caveolins.

In some embodiments, extracellular vesicles are analyzed, at the single extracellular vesicle level, for the presence of one or more of CD9, CD63 and CD81. In some embodiments, single extracellular vesicles are analyzed for the presence of CD9, CD63 and CD81.

In some embodiments, before extracellular vesicles are analyzed at the single extracellular vesicle level, extracellular vesicles are captured from conditioned medium before labeling with one or more antibodies that bind to a marker(s) of interest. In some embodiments, extracellular vesicles are captured on a solid phase, such as a chip or cartridge, before labeling with one or more antibodies that bind to marker(s) of interest. In some embodiments, the antibodies are labeled with a fluorophore.

Conditioned medium may in some embodiments be subjected to one or more further processing steps. For instance, conditioned media and/or extracellular vesicles in conditioned media may be removed, recovered, concentrated, enriched, isolated, purified, refrigerated, frozen, cryopreserved, lyophilized, sterilized, etc. Conditioned medium may also be pre-cleared or clarified by one or more centrifugation and/or filtration techniques. Extracellular vesicles may be enriched, purified, or further concentrated by centrifugation, ultracentrifugation, filtration, ultrafiltration, gravity, sonication, density-gradient ultracentrifugation, tangential flow filtration, size-exclusion chromatography, ion-exchange chromatography, affinity capture, polymer-based precipitation, or organic solvent precipitation, for example.

### EXPERIMENTAL

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for. It should be understood that these Examples are given by way of illustration only and are not intended to limit the scope of what the inventor regards as various embodiments of the present invention. Not all of the following steps set forth in each Example are required nor must the order of the steps in each Example be as presented.

### Example 1

### High-Throughput Media Development for Extracellular Vesicle Production from iPSC-Derived Cells

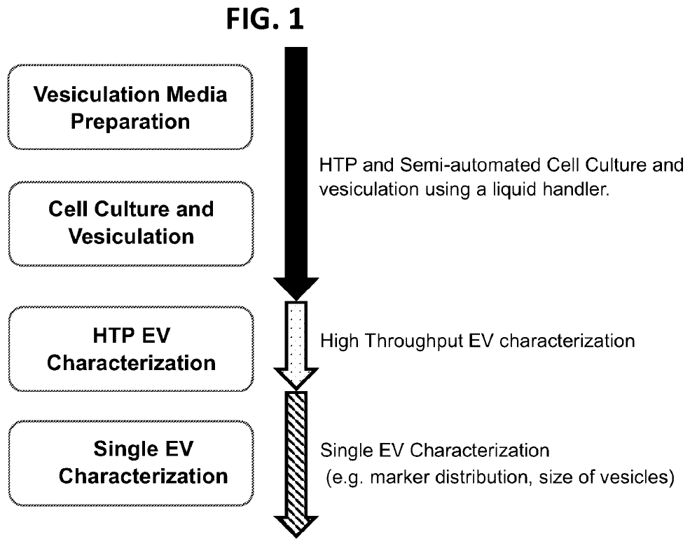

iPSC-derived cells were used as a model for extracellular vesicle production in a high-throughput assay for testing multiple vesiculation media formulations. This high-throughput, semi-automated assay allowed multiple different vesiculation media formulations to be produced and then tested simultaneously, while also reducing the length of time of the experiment. A flowchart of the various stages of an exemplary assay as conducted is shown in FIG. 1.

For preparing multiple different vesiculation media formulations for analysis, media blending was strategized using Design Expert software (StatEase), using a Design of Experiments (DoE) approach. Six different varieties of culture media (including commercially available culture media) were used as the starting media for blending. These six different varieties of media were blended to produce 31 different blends (M1-M31) of vesiculation media, using an automated liquid handler (a Biomek i7 automated liquid handler, manufactured by Beckman Coulter). The panel of the resulting 31 media blends was then tested as follows.

Following expansion culture of the iPSC-derived cells in multi-well plates, the expansion culture medium was removed from the cell culture wells, and replaced with either a benchmark control medium (“benchmark”), or one of the vesiculation media blends (M1-M31). Replicate wells were employed for the control and vesiculation media blends. The cells were then cultured to produce conditioned media containing extracellular vesicles. Removal of the expansion culture medium, replacement with the vesiculation media, vesiculation culturing, and removal of the conditioned media were all conducted in a semi-automated fashion (using a Biomek i7 automated liquid handler, manufactured by Beckman Coulter).

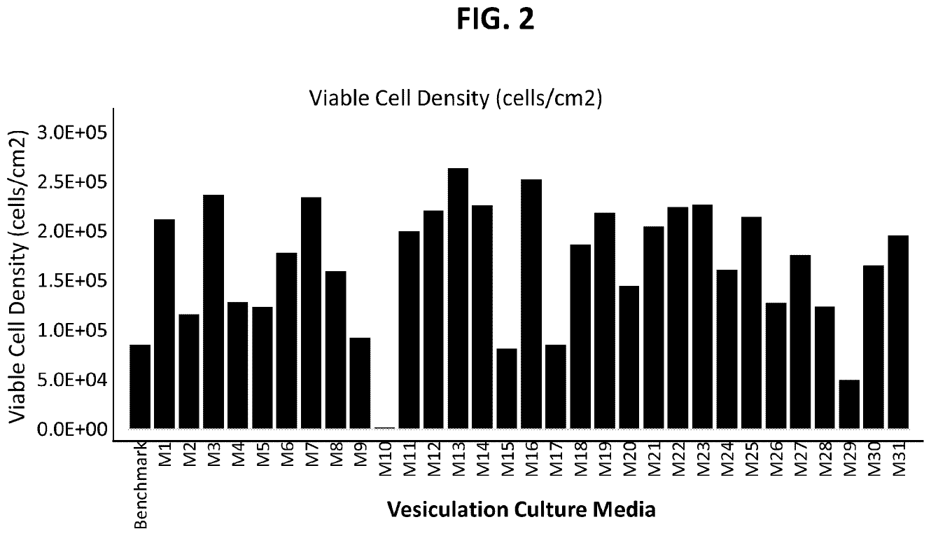

After the conditioned media were collected, the cultured cells were assessed for cell density and cell viability, in a semi-automated fashion. Briefly, live and dead cells were stained with AOPI (Acridine Orange and Propidium Iodide), and counted with an automated Cellaca Cell Counter, manufactured by Nexcelom. The results are shown in FIG. 2, which depicts the viable cell density after culturing with the control and M1-M31 vesiculation media formulations. As can be seen from FIG. 2, this procedure allowed to efficiently determine the effects of the different vesiculation media formulations on cell viability, and to identify the vesiculation media formulations that promoted cell growth.

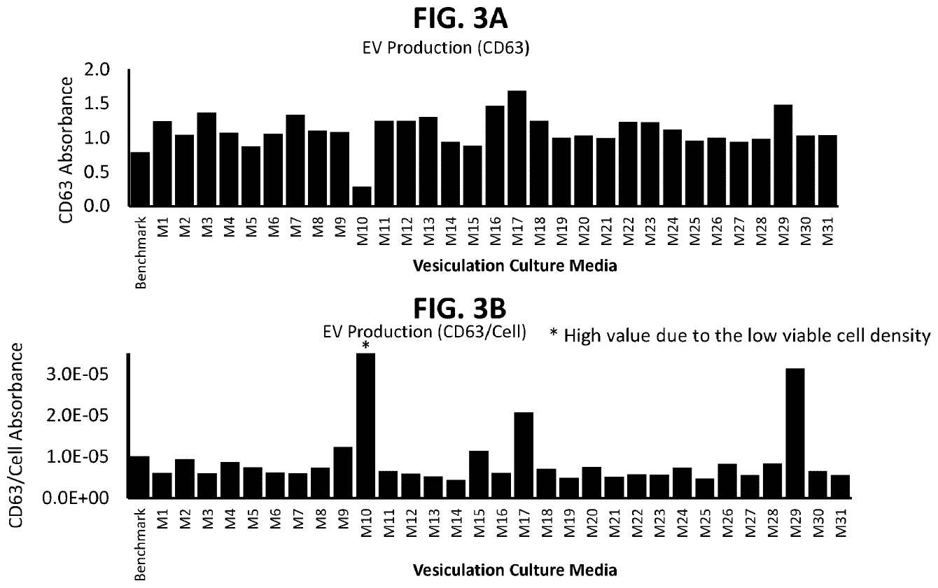

Additionally, the recovered conditioned media were then analyzed to determine extracellular vesicle production using the different vesiculation media. Extracellular vesicle production was determined using a high-throughput Tim4-based extracellular vesicle ELISA (EV ELISA) assay, wherein Tim4 was used to capture extracellular vesicles in the conditioned media (ELISA kit #297-79201, FUJIFILM Wako Pure Chemical). The captured extracellular vesicles were detected using an anti-CD63 antibody. The results are shown in FIGS. 3A and 3B. FIG. 3A depicts the data for total extracellular vesicle production, and FIG. 3B depicts extracellular vesicle production per cell. The total extracellular vesicle counts were estimated based on the amount of CD63 on the surface of extracellular vesicles. As can be seen from FIGS. 3A and 3B, this procedure allowed to efficiently determine the effects of the different vesiculation media formulations on extracellular vesicle production, and to identify the vesiculation media formulations that promoted extracellular vesicle production. FIGS. 3A and 3B also show that formulation M17 yielded more extracellular vesicles than the benchmark control media, and more than the other vesiculation media formulations tested.

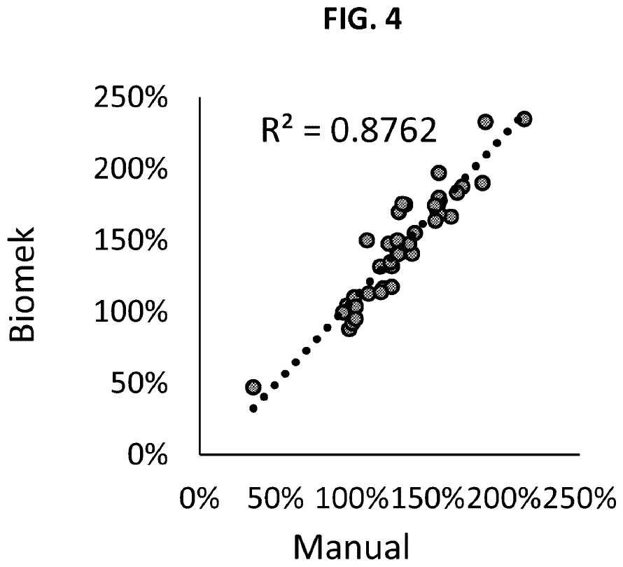

To determine the accuracy of the automated liquid handling steps described above, extracellular vesicle production using the automated liquid handling process was compared with equivalent steps performed by manual handling. Tim4-based extracellular vesicle ELISA assay described above was used for comparison. The results are shown in FIG. 4. The CD63 level in the benchmark media was set as 100%, and x and y axis show a relative EV production level. As shown in FIG. 4, extracellular vesicle production using the automated liquid handler closely correlated with extracellular vesicle production using manual handling, confirming the accuracy and robustness of the high-throughput, semi-automated, extracellular vesicle production process.

Next, extracellular vesicles in the conditioned media were characterized at the single extracellular vesicle level, using super resolution microscopy (Direct Stochastic Optical Reconstruction Microscopy (dSTORM)). Briefly, for each sample, 10 μL of conditioned medium was used as an input sample. Extracellular vesicles in the input sample were captured on a chip using the EV Profiler Kit (manufactured by ONi) and labeled with CD63, CD81, and CD9 fluorophore-conjugated antibodies included in the kit. Individual fluorophores were then localized using a Nanoimager S (manufactured by ONi). The acquired images were then processed and analyzed with CODI Software (https://oni.bio/applications/) and R.

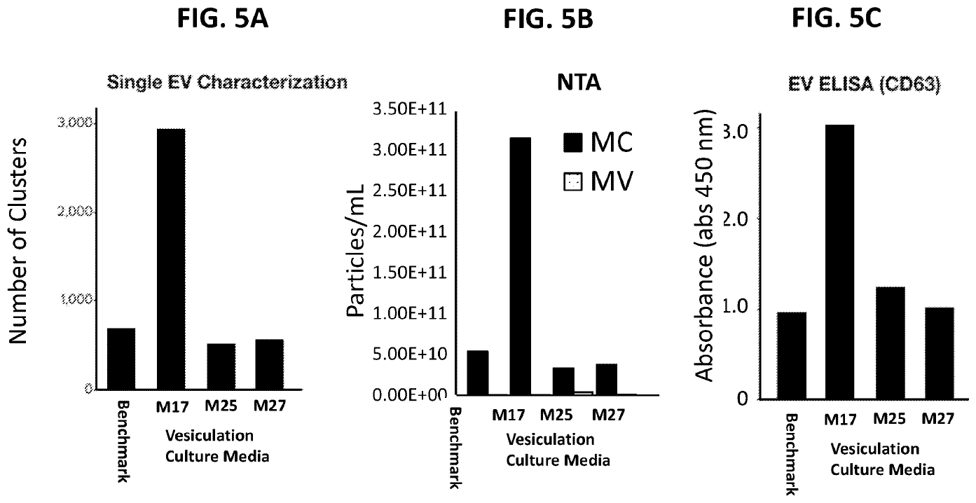

As shown in FIGS. 5A, 5B and 5C, the total extracellular vesicle counts determined from the single extracellular vesicle (dSTORM) analysis (FIG. 5A) and nanoparticle tracking analysis (NTA) (FIG. 5B) closely correlated with the CD63 expression level determined using the EV ELISA assay (FIG. 5C).

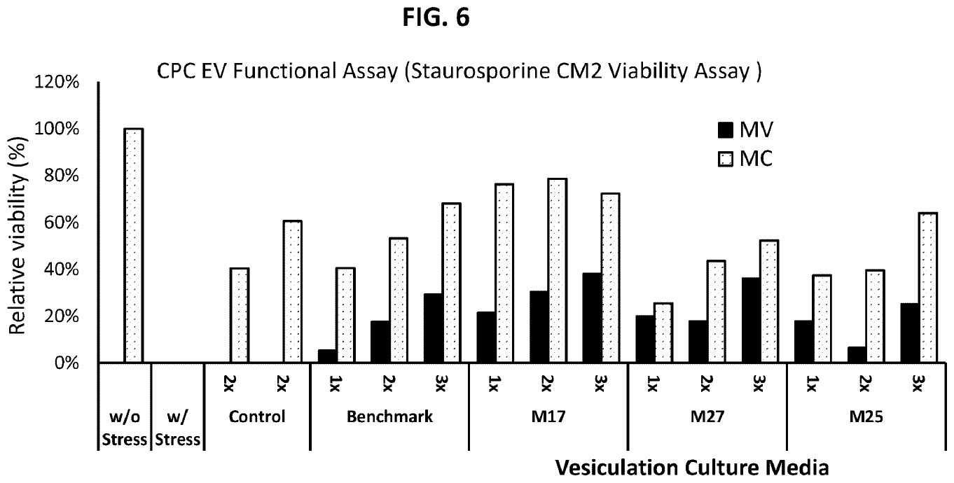

FIG. 6 shows the results of a human cardiomyocyte survival assay performed to evaluate EV function. EVs purified from conditioned media and mock control media using ultracentrifugation were used as input samples. The effects of the EV were tested in a cardiomyocyte survival assay as described in the International Patent Application Publication No. WO2022106890A1. EVs produced by the candidate vesiculation media were functional in the Staurosporine CM2 Viability Assay.

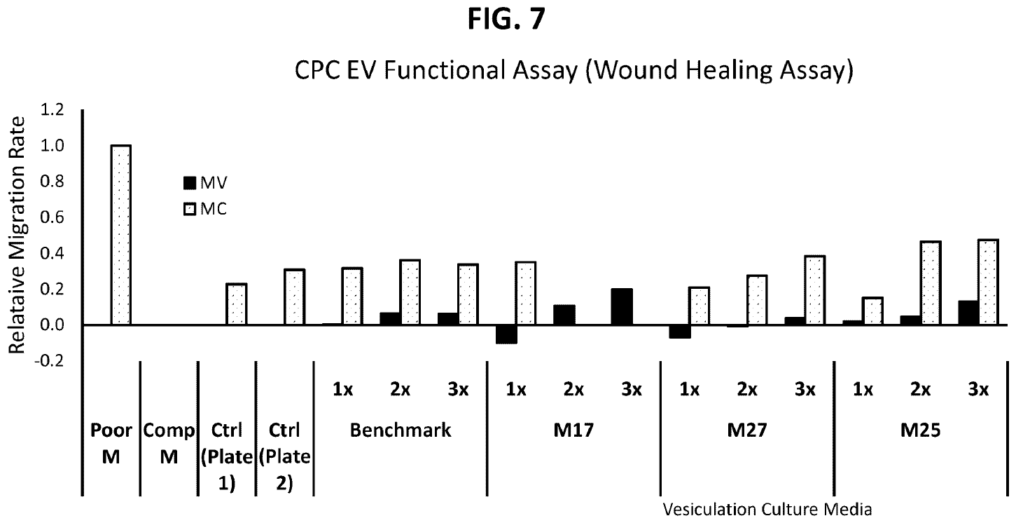

FIG. 7 shows the results of a HUVEC scratch wound healing assay performed to evaluate EV function. EVs purified from conditioned media and mock control media using ultracentrifugation were used as input samples. The scratch wound healing assay developed by Essen BioSciences for the IncuCyte® was employed according to the manufacturer's directions. The results show that EVs produced by the candidate vesiculation media were functional in the wound healing assay.

The above experiments demonstrate that using iPSC-derived cells as a model, vesiculation media that promoted cell growth and stimulated extracellular vesicle secretion as compared to a benchmark control medium could be identified through this high-throughput approach. These experiments further demonstrate the surprising effects that the culture media have on the cellular microenvironment, influencing not only cell expansion and cell quality, but also influencing extracellular vesicle yield, quality, and purity.

### Example 2

### High-Throughput Media Development for Extracellular Vesicle Production from Primary MSCs

Primary Mesenchymal Stem Cells (MSCs) were used as a model for extracellular vesicle production in a high-throughput assay for testing multiple vesiculation media formulations. This high-throughput, semi-automated assay allowed multiple different vesiculation media formulations to be produced and then tested simultaneously, while also reducing the length of time of the experiment. A flowchart of the various stages of an exemplary assay as conducted is shown in FIG. 1.

For preparing multiple different vesiculation media formulations for analysis, media blending was strategized using Design Expert software (StatEase), using a Design of Experiments (DoE) approach. Six different varieties of culture media (including commercially available culture media) were used as the starting media for blending. These six different varieties of media were blended to produce 46 different blends (M1-M46) of vesiculation media, using an automated liquid handler (a Biomek i7 automated liquid handler, manufactured by Beckman Coulter). The panel of the resulting 46 media blends was then tested as follows.

Following expansion culture of the MSCs in multi-well plates, the expansion culture medium was removed from the cell culture wells, and replaced with either a benchmark control medium (“benchmark 1 and benchmark 2”), or one of the vesiculation media blends (M1-M46). Replicate wells were employed for the control and some of the vesiculation media blends. The cells were then cultured to produce conditioned media containing extracellular vesicles. Removal of the expansion culture media, replacement with the vesiculation media, vesiculation culturing, and removal of the conditioned media were all conducted in a semi-automated fashion (using a Biomek i7 automated liquid handler, manufactured by Beckman Coulter).

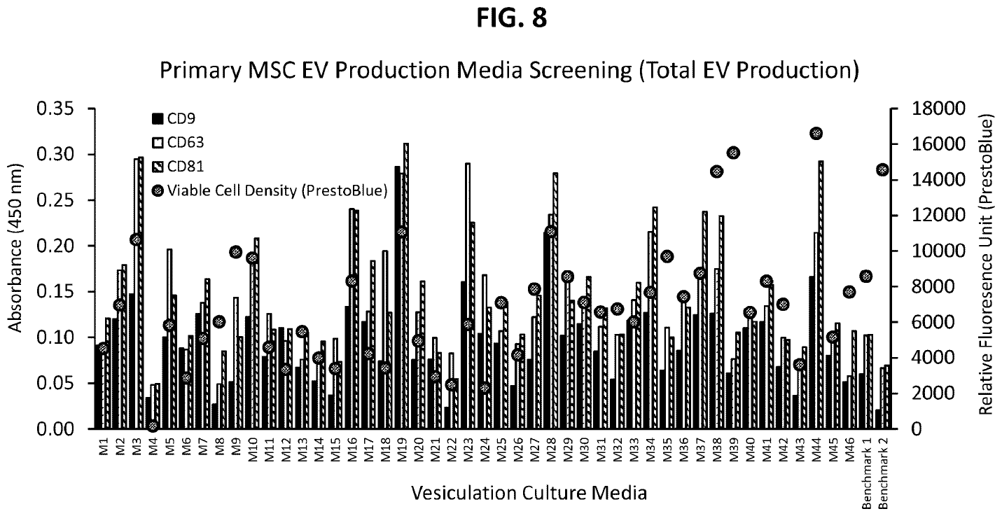

After the conditioned media was collected, the cultured cells were assessed for cell density in a semi-automated fashion. Briefly, live cells were stained with PrestoBlue™ Cell Viability Reagent (ThermoFisher Scientific), and fluorescence intensity was measured by a plate reader. The results are shown in FIG. 8, which depicts the viable cell density after culturing with the control and M1-M46 vesiculation media formulations (see the right vertical axis). As can be seen from FIG. 8, this procedure allowed to efficiently determine the effects of the different vesiculation media formulations on cell viability, and to identify the vesiculation media formulations that promoted cell growth.

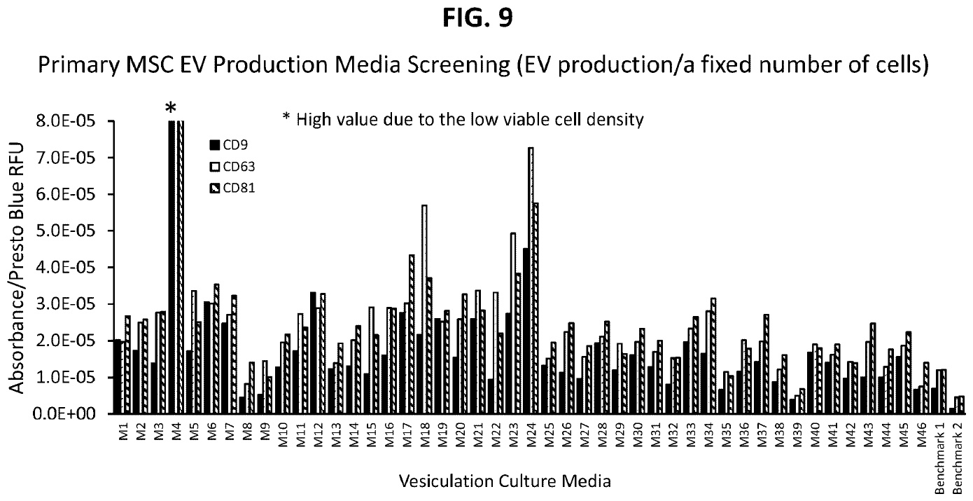

Additionally, the recovered conditioned media were then analyzed to determine extracellular vesicle production using the different vesiculation media. Extracellular vesicle production was determined using a high-throughput Tim4-based extracellular vesicle ELISA (EV ELISA) assay, wherein Tim4 was used to capture extracellular vesicles in the conditioned media, and the captured extracellular vesicles were detected using an anti-CD63, anti-CD9 and anti-CD81 antibody. The results are shown in FIGS. 8 and 9, which depict the data for total extracellular vesicle production, and extracellular vesicle production per fixed number of cells, respectively. The total extracellular vesicle counts were estimated based on the amount of CD63, CD9 and CD81 on the surface of extracellular vesicles. As can be seen from FIGS. 8 and 9, this procedure allowed to efficiently determine the effects of the different vesiculation media formulations on extracellular vesicle production, and to identify the vesiculation media formulations that promoted extracellular vesicle production. FIGS. 8 and 9 also show that some formulations, including formulation M3, yielded more extracellular vesicles than the benchmark control media, and more than the other vesiculation media formulations tested.

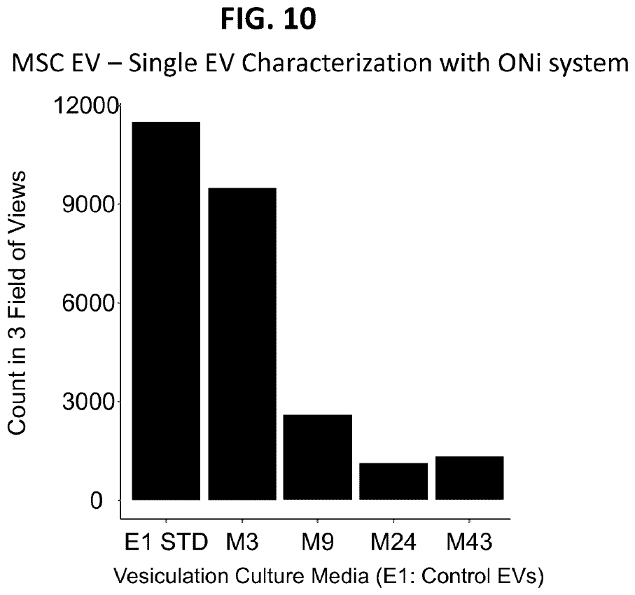

Next, extracellular vesicles in the conditioned media were characterized at the single extracellular vesicle level, using super resolution microscopy (Direct Stochastic Optical Reconstruction Microscopy (dSTORM)). Briefly, for each sample, 4.5 μL of purified EVs was used as an input sample. Extracellular vesicles in the input sample were captured on a chip using the EV Profiler Kit (manufactured by ONi), and then labeled with CD63, CD81, and CD9 antibodies before analysis. Individual fluorophores were then localized using a Nanoimager S (manufactured by ONi). The acquired images were then processed and analyzed with CODI Software (https://oni.bio/applications/) and R. The results are shown in FIG. 10.

Next, primary MSC cell culture and vesiculation was performed using T-150 flasks. Collected extracellular vesicles were analyzed using the EV ELISA assay.

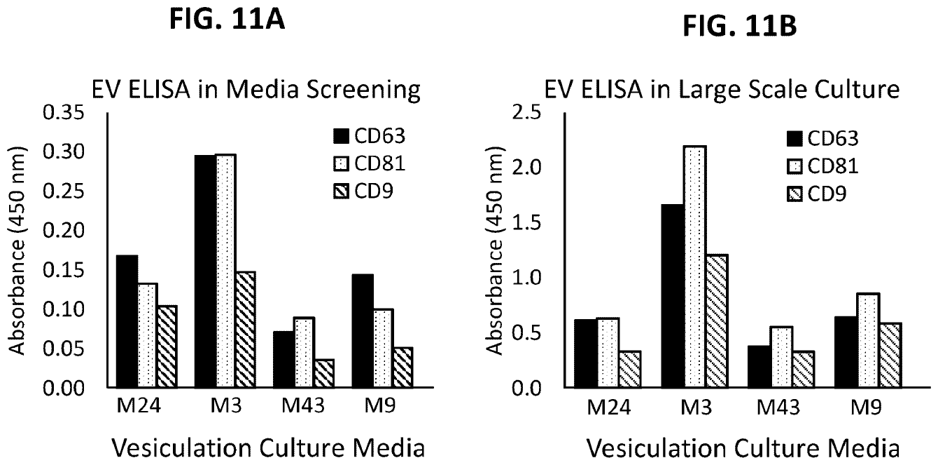

FIGS. 11A and 11B show CD63, CD9 and CD81 expression level determined using the extracellular vesicle ELISA assay for vesicles produced with the specified vesiculation media by the manual method in flasks (FIG. 11A) and by using the automated liquid handler in multi-well plates (FIG. 11B). As can be seen by comparing the results in FIGS. 10, 11A and 11B, the total extracellular vesicle counts determined from the single extracellular vesicle (dSTORM) analysis closely correlated with the CD63, CD9 and CD81 expression levels determined using the extracellular vesicle ELISA assay.

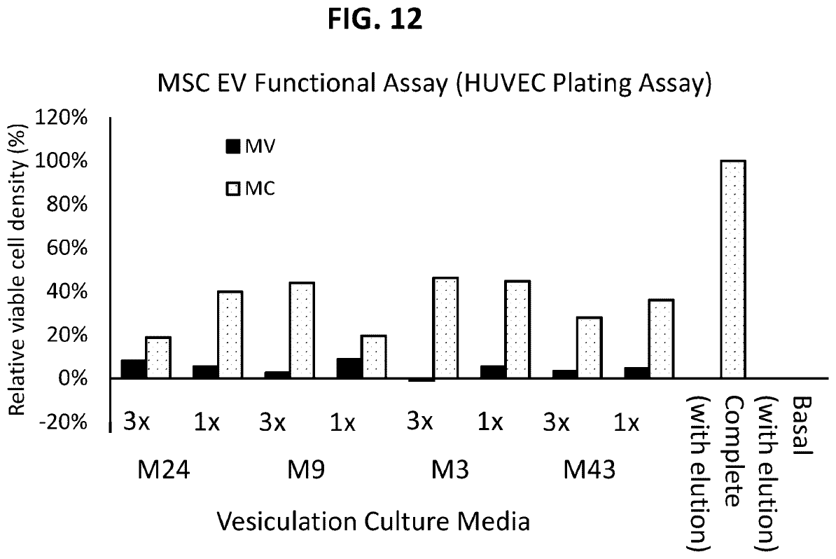

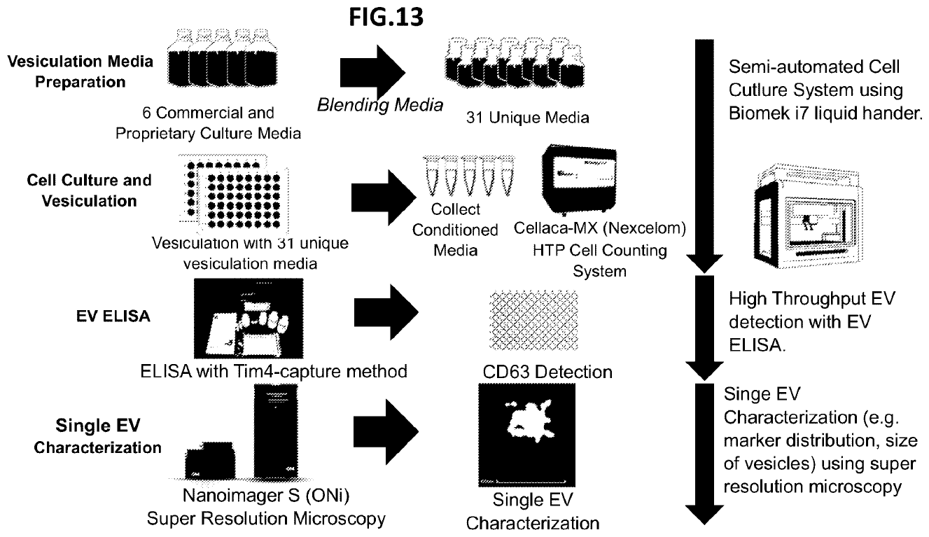

A HUVEC plating assay were performed to evaluate EV function. EVs purified from conditioned media and mock media using ultracentrifugation were used as input samples. As shown in FIG. 12, EVs purified from conditioned media stimulated the growth of HUVEC cells.

The above experiments therefore demonstrate that, using MSCs as a model, vesiculation media that promoted cell growth and stimulated extracellular vesicle secretion as compared to a benchmark control medium could be identified through this high-throughput approach. These experiments further demonstrate the surprising effects that the culture media have on the cellular microenvironment, influencing not only cell expansion and cell quality, but also influencing extracellular vesicle yield, quality, and purity.

What is claimed is:

1. A high-throughput method for analyzing, developing, and/or optimizing, a culture medium for extracellular vesicle production, said method comprising:
(a) culturing cells in a first culture medium, wherein cell division occurs during the culturing, and wherein said culturing is performed in multiplicate;
(b) after step (a) removing said first culture medium from the multiplicate cell cultures, adding different candidate vesiculation culture media to different cell cultures amongst said multiplicate cell cultures, and further culturing the multiplicate cell cultures to produce conditioned media containing extracellular vesicles;
(c) recovering, from the multiplicate cell cultures, either the conditioned media, the cells after the culturing of step (b), or both; and
(d) analyzing at least one property of either the extracellular vesicles in the recovered conditioned media, the recovered cells, or both,
wherein each of steps (a)-(c) is semi-automated using an automated liquid.

2. The method of claim 1, wherein at least one of steps (a)-(c) is fully-automated.

3. The method of claim 1, wherein each of steps (a)-(c) are semi-automated or fully-automated.

4. The method of any one of claims 1-3, wherein said cells are iPSC-derived cells and/or primary stem cells.

5. The method of any of claim 1-4 wherein the culturing is two-dimensional cell culture.

6. The method of claim 5, wherein said two-dimensional cell culture comprises culturing said cells on a surface of a culture vessel.

7. The method of claim 6, wherein said culture vessel surface is coated with a substance to promote cell adhesion.

8. The method of claim 7, wherein said substance to promote cell adhesion is vitronectin or fibronectin.

9. The method of any one of claims 1-8, wherein the multiplicate cell cultures are cultured within one or more multi-well plates or micro-well plates.

10. The method of any one of claims 1-9, wherein the different candidate vesiculation culture media in step (b) are obtained by blending or combining two or more culture media, and/or adding one or more additives or supplements to one or more culture media, to produce a panel of different candidate vesiculation culture media.

11. The method of claim 10, wherein the panel of different candidate vesiculation culture media is produced using an automated liquid handler.

12. The method of claim 11, wherein the panel of candidate vesiculation culture media is produced by blending or combining two or more different culture media from an initial selection of at least five different culture media.

13. The method of claim 11, wherein the panel of candidate vesiculation culture media is produced by blending or combining two or more culture media from an initial selection of at least ten different culture media.

14. The method of claim 11, wherein the panel of candidate vesiculation culture media is produced by blending or combining two or more culture media from an initial selection of at least twenty different culture media.

15. The method of claim 11, wherein the panel of candidate vesiculation culture media is produced by blending or combining two or more culture media from an initial selection of at least fifty different culture media.

16. The method of any one of claims 11-15, wherein the panel of candidate vesiculation culture media comprises at least ten candidate vesiculation culture media.

17. The method of claim 16, wherein the panel of candidate vesiculation culture media comprises at least twenty candidate vesiculation culture media.

18. The method of claim 16, wherein the panel of candidate vesiculation culture media comprises at least thirty candidate vesiculation culture media.

19. The method of claim 16, wherein the panel of candidate vesiculation culture media comprises at least fifty candidate vesiculation culture media.

20. The method of claim 16, wherein the panel of candidate vesiculation culture media comprises at least a hundred candidate vesiculation culture media.

21. The method of any one of claims 1-20, wherein said cells comprise progenitor cells.

22. The method of any one of claims 1-21, wherein said cells have previously been refrigerated or cryopreserved.

23. The method of any one of claims 1-22, wherein the at least one property of the recovered cells from step (c) analyzed is selected from the group consisting of the cell number, cell viability, cell density, morphologies of the cells, identity of the cells, karyotype of the cells, transcriptome of the cells, hypertrophy, cell health, cell adhesion, cell physiology, and/or ATP content.

24. The method of claim 23, wherein the at least one property is selected from the group consisting of cell number and cell viability.

25. The method of claim 24, wherein cell number and/or cell viability is measured using an automated cell counter.

26. The method of claim 24 or claim 25, wherein cell number and/or cell viability is determined by staining cells with at least one dye.

27. The method of claim 26, wherein the cells are stained with acridine orange and/or propidium iodide.

28. The method of any one of claims 1-22, wherein the at least one property of the extracellular vesicles analyzed is total extracellular vesicle number, extracellular vesicle number per cell, extracellular vesicle concentration, extracellular vesicle size, extracellular vesicle size distribution, protein concentration, protein profile concentration, RNA profile, potency, or marker expression.

29. The method of claim 28, wherein the at least one property of the extracellular vesicles analyzed is selected from total extracellular vesicle number, extracellular vesicle number per cell, extracellular vesicle size, and marker expression.

30. The method of claim 29, wherein the total extracellular vesicle number and/or extracellular vesicle number per cell is determined by measuring the expression of at least one marker present on extracellular vesicles.

31. The method of claim 30, wherein the marker is a tetraspanin.

32. The method of claim 31, wherein the tetraspanin is selected from the group consisting of CD9, CD63 and CD81.

33. The method of claim 31, wherein the tetraspanin is CD63.

34. The method of any one of claims 29-33, wherein the marker is detected using an immunoassay.

35. The method of claim 34, wherein the immunoassay is ELISA.

36. The method of claim 35, wherein the ELISA is a Tim4-capture ELISA.

37. The method of claim 36, wherein the ELISA is a Tim4-capture ELISA that detects CD63 expression.

38. The method of any one of claims 23-37, wherein the measurement of the at least one property is at least partially automated.

39. The method of claim 28, wherein the at least one property of the extracellular vesicles analyzed is analyzed at the single extracellular vesicle level.

40. The method of claim 39, wherein the at least one property analyzed at the single extracellular vesicle level is marker expression.

41. The method of claim 39 or 40, wherein the analysis is conducted using super resolution microscopy.

42. The method of claim 41 wherein the super resolution microscopy is Direct Stochastic Optical Reconstruction Microscopy (dSTORM).

43. The method of any of claims 40-42, wherein the marker expression analyzed comprises analysis of the expression of at least one tetraspanin.

44. The method of claim 43, wherein the tetraspanin is selected from the group consisting of CD9, CD63 and CD81.

45. The method of any one of claims 39, 41 and 42, wherein the size of individual extracellular vesicles is analyzed.

46. The method of any one of claims 42-45 wherein the analysis is used to analyze extracellular vesicle subpopulations.

47. The method of claim 1, wherein in said method,
the culturing comprises culturing said cells on a surface of a culture vessel, said culture vessel being a multi-well plate or a micro-well plate,
the different candidate vesiculation culture media in step (b) are obtained by blending or combining two or more culture media together, from an initial selection of at least two different culture media, to produce a panel of different candidate vesiculation culture media,
said method comprises, in step (d), analyzing cell number and cell viability using an automated cell counter,
said method further comprises, in step (d), measuring the total extracellular vesicle number and/or the extracellular vesicle number per cell, by measuring the expression of at least one marker present on extracellular vesicles by a high-throughput immunoassay, and
said method further comprises, in step (d), analyzing marker expression and/or vesicle size of individual extracellular vesicles by super resolution microscopy.

48. The method of claim 47, wherein the immunoassay is a Tim4-capture ELISA.

49. The method of claim 48, wherein the ELISA is a Tim4-capture ELISA that detects CD63 expression.

50. The method of any one of claims 47-49, wherein the analysis comprises analyzing one or more of CD9, CD63 and CD81 expression by said super resolution microscopy.

51. The method of claim 50, wherein the analysis comprises analyzing CD9, CD63 and CD81 expression by said super resolution microscopy.

52. The method of any one of claims 50 and 51, further comprising analyzing the size of individual extracellular vesicles by said super resolution microscopy.

53. The method of any one of claims 1-52, further comprising selecting a vesiculation media from among the candidate vesiculation media based on the results of the analysis of step (d).

54. The method of claim 53, wherein the selected media provides an improvement in one or more of cell growth, cell viability, total extracellular vesicle number, extracellular vesicle number per cell, marker expression on extracellular vesicles, and extracellular vesicle size, as compared to a control benchmark medium, or an unblended medium.

55. A vesiculation media selected by the method of claim 53 or 54.

56. A system for performing the method of any one of claims 1-55, wherein said system comprises one or more of an automated liquid handler, an automated cell counter, an immunoassay kit, and a super resolution microscope.

57. The system of claim 56, wherein the immunoassay kit is an ELISA kit.

58. The method of any one of claims 47-57, wherein the panel of candidate vesiculation culture media is produced by blending or combining three or more different culture media.

59. The method of any one of claims 47-57, wherein the panel of candidate vesiculation culture media is produced by blending or combining four or more different culture media.

60. The method of any one of claims 47-59, wherein the blending or combining of the two or more culture media together is from an initial selection of at least five different culture media.

61. The method of any one of claims 47-59, wherein the analysis by super resolution microscopy includes immobilizing extracellular vesicles on at least one of a coverslip and a microscopy channel slide.

62. The method of any one of claims 47-61, wherein the analysis by super resolution microscopy includes detecting the at least one marker by using a fluorescent antibody.
